(19) United States
(12) Patent Application Publication
(10) Pub. No.: US 2017/0121774 A1
(43) Pub. Date: May 4, 2017

(54) METHODS AND COMPOSITIONS FOR ASSESSING PREDICTING RESPONSIVENESS TO A TNF INHIBITOR

(71) Applicant: The Board of Trustees of the Leland Stanford Junior University, Stanford, CA (US)

(72) Inventors: Francesco Vallania, San Francisco, CA (US); Purvesh Khatri, Menlo Park, CA (US)

(21) Appl. No.: 15/336,590

(22) Filed: Oct. 27, 2016

Related U.S. Application Data
(60) Provisional application No. 62/247,665, filed on Oct. 28, 2015.

(51) Int. Cl. C12Q 1/68; G01N 33/68

(52) U.S. Cl. CPC C12Q 1/6883; G01N 33/6893; C12Q 2600/106; C12Q 2600/158; G01N 2333/726; G01N 2333/5431; G01N 2800/065; G01N 2800/52

(57) ABSTRACT

Methods, systems (e.g., computer systems), compositions, and kits are provided for predicting whether an individual will respond to treatment with a TNF inhibitor, for determining a treatment regimen for an individual (e.g. a therapy that does or does not include administration of a TNF inhibitor), and for treating an individual. The subject methods include measuring an expression level of an RGS1 expression product and/or an expression level of an IL11 expression product in a biological sample from an individual. In some cases, the methods include a step of calculating a TNF inhibitor signature score from measured expression levels (e.g., calculating a geometric mean of the expression levels of an RGS1 expression product and an IL11 expression product). After comparing measured expression levels and/or a calculated TNF inhibitor signature score with a reference, one can predict whether an individual will respond to treatment with a TNF inhibitor.

### CROSS REFERENCE

This application claims benefit U.S. Provisional Patent Application No. 62/247,665, filed Oct. 28, 2015, which application is incorporated herein by reference in its entirety.

### FEDERALLY SPONSORED RESEARCH AND DEVELOPMENT

This invention was made with Government support under contracts AI057229, AI089859, AI109662, AI117925, and HL120001 awarded by the National Institutes of Health. The Government has certain rights in the invention.

### INTRODUCTION

Predicting drug response before treatment is a fundamental goal in modern medicine. Patient variability in drug response can lead to deleterious side effects without the expected benefits. This not only results in harming the patient but also dramatically increases health-care costs.

The treatment of autoimmune disorders with TNF inhibiting drugs would greatly benefit from advances in drug response prediction. Autoimmune disorders are common diseases, affecting ˜8% of the population in the United States, and represent a significant social and health-care burden. TNF inhibitors (TNFi) are a class of drugs that suppress the response to Tumor Necrosis Factor alpha (TNFalpha), a component of the inflammatory response. TNFi are used to treat autoimmune and immune-mediated disorders. Unfortunately, only ˜20 to 45% of patients with autoimmunity develop a sustained drug response after TNFi treatment. Furthermore, annual TNFi treatment is expensive and TNFi treatment can cause significant side effects.

There is a need in the art for methods that use a small set of biomarkers to guide therapy by predicting TNFi response. For example, such methods could result in reduced health-care costs and could spare non-responsive patients from experiencing side effects. Because TNFi are used to treat a variety of different disorders, such methods would be broadly applicable.

### SUMMARY

Methods, systems (e.g., computer systems), compositions, and kits are provided for predicting whether an individual will respond to treatment with a TNF inhibitor, for determining a treatment regimen for an individual (e.g. a therapy that does or does not include administration of a TNF inhibitor), and for treating an individual. The subject methods include measuring an expression level of an RGS1 expression product and/or an expression level of an IL11 expression product in a biological sample from an individual. In some cases, the methods include a step of calculating a TNF inhibitor signature score from measured expression levels (e.g., calculating a geometric mean of the expression levels of an RGS1 expression product and an IL11 expression product). After comparing measured expression levels and/or a calculated TNF inhibitor signature score with a reference, one can predict whether an individual will respond to treatment with a TNF inhibitor.

The subject methods (e.g., for predicting whether an individual will respond to treatment with a TNF inhibitor) include measuring an expression level of an RGS1 expression product and/or an expression level of an IL11 expression product in a biological sample from an individual. In some cases, the methods include a step of calculating a TNF inhibitor signature score from measured expression levels (e.g., calculating a geometric mean of the expression levels of an RGS1 expression product and an IL11 expression product). In some cases calculating includes the use of a processor configured to calculate said geometric mean. In some cases the RGS1 expression product is an RNA encoding the RGS1 protein and in some cases the RGS1 expression product is the RGS1 protein. In some cases the IL11 expression product is an RNA encoding the IL11 protein and in some cases the IL11 expression product is the IL11 protein. In some cases, the measuring step includes an assay selected from: quantitative RT-PCR, microarray, and nucleic acid sequencing. In some cases, the measuring step includes an assay selected from: ELISA, Western blot, mass spectrometry, and flow cytometry. In some cases, such methods include a step of providing a prediction (e.g., that the individual will or will not respond to treatment with a TNF inhibitor). In some cases, the subject methods include a step of generating a report. In some cases, the report includes a measured expression level of an RGS1 expression product and/or an IL11 expression product. In some cases, the report further includes a reference value (e.g. which can be used for providing a prediction). In some cases, the report includes a calculated TNF inhibitor signature score, and in some cases the report further includes a reference value for the TNF inhibitor signature score.

Thus, in some embodiments, a method of for predicting whether an individual will respond to treatment with a TNF inhibitor includes (a) measuring an expression level of an RGS1 expression product and an expression level of an IL11 expression product in a biological sample from an individual; (b) calculating a geometric mean of said measured expression levels to obtain a TNF inhibitor signature score for the individual; and (c) generating a report that includes the TNF inhibitor signature score and a reference value for the TNF inhibitor signature score.

In some cases, any of the above subject methods (e.g., for predicting whether an individual will respond to treatment with a TNF inhibitor) further include: (i) determining that the expression level(s) (or TNF inhibitor signature score) is less than or equal to the reference value, and predicting that the individual will respond to treatment with a TNF inhibitor; or (ii) determining that the expression level(s) (or TNF inhibitor signature score) is greater than or equal to the reference value, and predicting that the individual will not respond to treatment with a TNF inhibitor. In some cases, the step of determining that the expression level(s) (or TNF inhibitor signature score) is less than or equal to the reference value includes, after said determining, a step of treating the individual with a TNF inhibitor; and the step of determining that the expression level(s) (or TNF inhibitor signature score) is greater than or equal to the reference value includes, after said determining, a step of treating the individual with a therapy that does not include administration of a TNF inhibitor.

In some cases, any of the above subject methods (e.g., for predicting whether an individual will respond to treatment with a TNF inhibitor) further include (i) after said calculating, determining that the expression level(s) (or TNF inhibitor signature score) is less than or equal to the reference value, where the report includes a prediction that the individual will respond to treatment with a TNF inhibitor; or (ii) after said calculating, determining that the expression level(s) (or TNF inhibitor signature score) is greater than or equal to the reference value, where the report includes a prediction that the individual will not respond to treatment with a TNF inhibitor.

In some embodiments, a subject method is a method of treating an individual in need thereof, and the method includes: (a) measuring an expression level of an RGS1 expression product and an expression level of an IL11 expression product in a biological sample from an individual; (b) calculating the geometric mean of said measured expression levels to obtain a TNF inhibitor signature score; and either: (i) determining that the TNF inhibitor signature score is less than or equal to a reference value, and treating the individual with a TNF inhibitor; or (ii) determining that the TNF inhibitor signature score is greater than or equal to a reference value, and treating the individual with a therapy that does not include administration of a TNF inhibitor.

In some embodiments, a subject method is a method of treating an individual with inflammatory bowel disease and/or psoriasis, and the method includes: measuring an expression level of an RGS1 expression product in a biological sample from the individual, and either (i) determining that said expression is less than or equal to a reference value, and treating the individual with a TNF inhibitor, or (ii) determining that said expression is greater than or equal to a reference value, and treating the individual with a therapy that does not include administration of a TNF inhibitor.

A subject individual in any of the above methods (e.g, methods for predicting whether an individual will respond to treatment with a TNF inhibitor, methods of treating an individual in need thereof) can have an autoimmune and/or immune-mediated disorder, including but not limited to: inflammatory bowel disease (IBD), psoriasis, plaque psoriasis, psoriatic arthritis, Crohn's disease (CD), ulcerative colitis (UC), rheumatoid arthritis (RA), ankylosing spondylitis, hidradenitis suppurativa, and refractory asthma. Thus, TNF inhibitors can be used to treat autoimmune and immune-mediated disorders (e.g., inflammatory bowel disease (IBD), psoriasis, plaque psoriasis, psoriatic arthritis, Crohn's disease (CD), ulcerative colitis (UC), rheumatoid arthritis (RA), ankylosing spondylitis, hidradenitis suppurativa, and refractory asthma). One or more TNF inhibitors can be used to treat diseases (disorders) that include, but are not limited to: inflammatory bowel disease (IBD), psoriasis, plaque psoriasis, psoriatic arthritis, Crohn's disease (CD), ulcerative colitis (UC), rheumatoid arthritis (RA), ankylosing spondylitis, hidradenitis suppurativa, and refractory asthma. In some cases, the individual has inflammatory bowel disease and/or psoriasis. In some cases, the individual has inflammatory bowel disease. In some cases, the individual has psoriasis. In some cases, the biological sample is a biopsy. In some cases, the biological sample is a tissue sample collected from a site of inflammation.

Also provided are systems (e.g., computer systems), compositions, and kits for practicing the subject methods. For example, a subject system can include (I) a biomolecule analyzing system that includes: a detector for measuring an expression level of an RGS1 expression product and an expression level of an IL11 expression product, wherein the detector is coupled to a computer system; and (II) the computer system, that includes (i) a processor; and (ii) memory operably coupled to the processor, wherein the memory programs the processor to: (a) receive assay data from the detector of the biomolecule analyzing system, wherein the assay data includes the expression level of the RGS1 expression product and the expression level of the IL11 expression product; (b) calculate a geometric mean of said expression levels to obtain a TNF inhibitor signature score for the individual; and (c) generate a report that includes the TNF inhibitor signature score and a reference value for the TNF inhibitor signature score. In some cases, the memory programs the processor to compare the TNF inhibitor signature score to said reference value and to include in the report a prediction as to whether the individual is responsive to a TNF inhibitor.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1. Depiction of scheme for generating a disease-independent signature for a drug-response prediction.

FIG. 2A-2C. (FIG. 2A) Performance of disease-centered signature in discovery cohorts. (FIG. 2A) Separation between responders and non-responders at baseline in GSE12251 (p=1.775e-05). (FIG. 2B) Separation between responders and non-responders at baseline, and healthy controls (responders vs non-responders p=1.656e-03, responders vs controls p=6.66e-04, non-responders vs controls p=2.680e-05). (FIG. 2C) Prediction accuracy for baseline response in GSE12251 and GSE14580 measured by ROC curves.

FIG. 3A-3B. Performance of disease-centered signature in IBD validation cohort (GSE16879). (FIG. 3A) Separation between responders and non-responders at baseline and after therapy, and healthy controls in both colon (circles) and ileum (triangles). (FIG. 3B) Prediction accuracy for baseline response in colon and ileum measured by ROC curves.

FIG. 4A-4C. Selection of disease-independent biomarkers from disease-centered signature (FIG. 4A) Correlation plot between summary effect size values of disease-centered Infliximab response signature (y-axis) and Ulcerative Colitis (x-axis). Yellow points indicate genes that show a significant effect size for Ulcerative Colitis but not for Infliximab response; blue points indicate genes with a significant effect size for Infliximab response but nor for Ulcerative Colitis; green points are genes with significant effect sizes in both Infliximab response and Ulcerative Colitis. (FIG. 4B, FIG. 4C) Forest plots of IL11 and RGS1, the two genes identified as disease-independent biomarkers. Blue box represents effect size of the gene for a particular study with error bars indicating the 95% confidence interval. The orange rhomboid shape indicates the gene's summary effect size with length indicative of its 95% confidence interval.

FIG. 5A-5B. Performance of disease-independent signature in in IBD validation cohort (GSE16879). (FIG. 5A) Prediction accuracy of disease-centered signature (same as FIG. 3B) (FIG. 5B) Prediction accuracy of disease-independent signature for ileum and colon biopsies. Performance is measured by ROC.

FIG. 6A-6C. Performance of disease-independent signature in Psoriasis validation cohort (GSE11903). (FIG. 6A) Separation of responders (blue points) from non-responders (red points) using the disease-centered signature (p>0.05). (FIG. 6B) Separation of responders (blue points) from non-responders (red points) using the disease-independent signature (p<=0.05). (FIG. 6C) Prediction accuracy for disease-centered (green line) and disease-independent (blue line) measured by ROC curves.

FIG. 7A-7B. Significance assessment of disease-independent signature in Psoriasis validation cohort (GSE11903). (FIG. 7A) Prediction accuracy distribution of all possible gene pairs from the disease-centered signature expressed as AUC. Dashed line indicates the AUC obtained by using the disease-independent signature (p=0.00140). (FIG. 7B) Prediction accuracy distribution of 100,000 Monte-Carlo sampled gene pairs from the whole genome expressed as AUC. Dashed line indicates the AUC obtained by using the disease independent signature (p=0.00727).

FIG. 8. ROC plots on the IBD and Psoriasis validation datasets comparing the performance of biomarkers RGS1 and IL11 in combination (e.g., using the geometric mean), compared to the performance of either biomarker alone.

FIG. 9. Immunostaining (using an anti-RGS1 antibody) of paraffin embedded colon biopsy from a patient that is responsive to treatment with TNFα. Scale bar is 100 μm.

### DETAILED DESCRIPTION

Methods are provided for predicting whether an individual will respond to treatment with a TNF inhibitor, for determining a treatment regimen for an individual (e.g. a therapy that does or does not include administration of a TNF inhibitor), and for treating an individual (e.g., administering a TNF inhibitor to an individual or instead providing a therapy that does not include administration of a TNF inhibitor to the individual).

The subject methods include measuring an expression level of an RGS1 expression product and/or an expression level of an IL11 expression product in a biological sample from an individual. In some cases, the methods include a step of calculating a TNF inhibitor signature score from measured expression levels (e.g., calculating a the geometric mean of the expression levels of an RGS1 expression product and an IL11 expression product). In some cases calculating includes the use of a processor configured to calculate said geometric mean. In some cases the RGS1 expression product is an RNA encoding the RGS1 protein and in some cases the RGS1 expression product is the RGS1 protein. In some cases the IL11 expression product is an RNA encoding the IL11 protein and in some cases the IL11 expression product is the IL11 protein.

In some cases, the subject methods include a step of providing a prediction (e.g., that the individual will or will not respond to treatment with a TNF inhibitor). In some cases, the subject methods include a step of generating a report. In some cases, the report includes a measured expression level of an RGS1 expression product and/or an IL11 expression product. In some cases, the report further includes a reference value (e.g. which can be used for providing a prediction). In some cases, the report includes a calculated TNF inhibitor signature score, and in some cases the report further includes a reference value for the TNF inhibitor signature score.

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order that is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g., polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Methods

Aspects of the disclosure include methods of predicting whether an individual (e.g., an individual with inflammatory bowel disease and/or psoriasis) will respond to treatment with a TNF inhibitor, methods of determining a treatment regimen for an individual (e.g., an individual with inflammatory bowel disease and/or psoriasis), and methods of treating an individual (e.g., an individual in need thereof, an individual with inflammatory bowel disease and/or psoriasis, etc.).

### Diseases/Disorders and TNF Inhibitors

The terms “TNF inhibitor”, “TNFi”, “anti-TNF drugs”, and “anti-TNF agent” are used interchangeably herein to refer to an agent that selectively targets (interferes with the function of) tumor necrosis factor (TNF) and can be used to treat diseases associated with TNF activity, which can cause inflammation. Examples of TNF inhibitors (anti-TNF agents) include but are not limited to: anti-TNF antibodies, binding fragments from anti-TNF antibodies, engineered TNF-binding proteins (e.g., TNF receptor fusion proteins), anti-TNF small molecules, and the like. Examples of TNF inhibitors (anti-TNF agents) include but are not limited to: Infliximab, Adalimumab, Certolizumab pegol (CDP-870), Etarnecept, Golimumab, Pegsunercept, and the like.

As noted above, TNF inhibitors can be used to treat diseases associated with TNF activity. Such diseases include autoimmune and immune-mediated disorders including, but not limited to: inflammatory bowel disease (IBD), psoriasis, plaque psoriasis, psoriatic arthritis, Crohn's disease (CD), ulcerative colitis (UC), rheumatoid arthritis (RA), ankylosing spondylitis, hidradenitis suppurativa, and refractory asthma. Thus, TNF inhibitors can be used to treat autoimmune and immune-mediated disorders (e.g., inflammatory bowel disease (IBD), psoriasis, plaque psoriasis, psoriatic arthritis, Crohn's disease (CD), ulcerative colitis (UC), rheumatoid arthritis (RA), ankylosing spondylitis, hidradenitis suppurativa, and refractory asthma). One or more TNF inhibitors can be used to treat diseases (disorders) that include, but are not limited to: inflammatory bowel disease (IBD), psoriasis, plaque psoriasis, psoriatic arthritis, Crohn's disease (CD), ulcerative colitis (UC), rheumatoid arthritis (RA), ankylosing spondylitis, hidradenitis suppurativa, and refractory asthma.

As such, in some cases, an individual of the subject methods (e.g., an individual from whom a biological sample is obtained) has an autoimmune and/or immune-mediated disorder. In some cases, an individual of the subject methods (e.g., an individual from whom a biological sample is obtained) has a disorder (disease) selected the group consisting of: inflammatory bowel disease (IBD), psoriasis, plaque psoriasis, psoriatic arthritis, Crohn's disease (CD), ulcerative colitis (UC), rheumatoid arthritis (RA), ankylosing spondylitis, hidradenitis suppurativa, and refractory asthma.

In some cases, an individual of the subject methods (e.g., an individual from whom a biological sample is obtained) has inflammatory bowel disease (IBD) and/or psoriasis. In some cases, an individual of the subject methods (e.g., an individual from whom a biological sample is obtained) has inflammatory bowel disease (IBD). In some cases, an individual of the subject methods (e.g., an individual from whom a biological sample is obtained) has Crohn's disease (CD). In some cases, an individual of the subject methods (e.g., an individual from whom a biological sample is obtained) has ulcerative colitis (UC). In some cases, an individual of the subject methods (e.g., an individual from whom a biological sample is obtained) has psoriasis. In some cases, an individual of the subject methods (e.g., an individual from whom a biological sample is obtained) has plaque psoriasis. In some cases, an individual of the subject methods (e.g., an individual from whom a biological sample is obtained) has psoriatic arthritis.

Biomarkers of Responsiveness to Treatment with TNF Inhibitors

Aspects of the disclosure include measuring an expression level of one or more biomarkers (e.g., an expression level of an RGS1 expression product and/or an expression level of an IL11 expression product) in a biological sample from an individual. A biomarker is a molecular entity (e.g., an expression product such as mRNA, protein, etc.) whose representation in a sample correlates (either positively or negatively) with a particular state. For example, the biomarkers described herein correlate with whether an individual is responsive or non-responsive to TNF inhibitor treatment (i.e., therapy that includes the administration of one or more TNF inhibitors). Such biomarkers are differentially represented (i.e. represented at a different level) in a sample from an individual who is non-responsive to treatment with a TNF inhibitor (i.e., TNF inhibitor therapy) relative to an individual who is responsive to such treatment.

As demonstrated in the examples of the present disclosure, the inventors have identified RGS1 and IL11 as biomarkers (markers) that are associated with increased likelihood that a given individual will be non-responsive to treatment with a TNF inhibitor. The expression levels of RGS1 and/or IL11 (expression level of an expression product of RGS1 and/or IL11), e.g., alone or in combination, can provide a prediction (e.g., a determination) as to whether an individual will respond to treatment with a TNF inhibitor (e.g., as to whether the individual is a responder or non-responder). The term “expression product”.

RGS1 is also known in the art as “regulator of G-protein signaling 1”, 1R20, BL34, HEL-S-87, IER1, and IR20. The amino acid sequence of human RGS1 is:

RGS1 (NP_002913.3)
(SEQ ID NO: 1)
MRAAAISTPKLDKMPGMFFSANPKELKGTTHSLLDDKMQKRRPKTFGM
DMKAYLRSMIPHLESGMKSSKSKDVLSAAEVMOWSQSLEKLLANQTGQ
NVFGSFLKSEFSEENIEFWLACEDYKKTESDLLPCKAEEIYKAFVHSD
AAKQINIDFRTRESTAKKIKAPTPTCFDEAQKVIYTLMEKDSYPRFLK
SDIYLNLLNDLQANSLK

A human mRNA encoding the above protein is:

RGS1 (NM_002922.3) (presented in DNA form here):
(SEQ ID NO: 2)
GCCTGTCTGCATTCTACTATATAAAGCAGCAGAGACGTTGACTAGCGC
ATATTTGCTAAGAGCACCATGCGCGCAGCAGCCATCTCCACTCCAAAG
TTAGACAAAATGCCAGGAATGTTCTTCTCTGCTAACCCAAAGGAATTG
AAAGGAACCACTCATTCACTTCTAGACGACAAAATGCAAAAAAGGAGG
CCAAAGACTTTTGGAATGGATATGAAAGCATACCTGAGATCTATGATC
CCACATCTGGAATCTGGAATGAAATCTTCCAAGTCCAAGGATGTACTT
TCTGCTGCTGAAGTAATGCAATGGTCTCAATCTCTGGAAAAACTTCTT
GCCAACCAAACTGGTCAAAATGTCTTTGGAAGTTTCCTAAAGTCTGAA
TTCAGTGAGGAGAATATTGAGTTCTGGCTGGCTTGTGAAGACTATAAG
AAAACAGAGTCTGATCTTTTGCCCTGTAAAGCAGAAGAGATATATAAA
GCATTTGTGCATTCAGATGCTGCTAAACAAATCAATATTGACTTCCGC
ACTCGAGAATCTACAGCCAAGAAGATTAAAGCACCAACCCCCACGTGT
TTTGATGAAGCACAAAAAGTCATATATACTCTTATGGAAAAGGACTCT
TATCCCAGGTTCCTCAAATCAGATATTTACTTAAATCTTCTAAATGAC
CTGCAGGCTAATAGCCTAAAGTGACTGGTCCCTGGCTGAAGGGAATTA
ACAGATAGTATCAAGCGCAGAAGGAATGTGCCAGTATGGCTCCCTGGG
TGAACAGCTTGGCCTTTTTTGGGTGTCTTGACAGGCCAAGAAGAACAA
ATGACTCAGAATGGATTAACATGAAAGTTATCCAGGCGCAGAGTTGAA
GAAGCATAAGCAAGACAAAAACAGAGAGACCGCAGAAGGAGGAAGATA
CTGTGGTACTGTCATAAAAAACAGTGGAGCTCTGTATTAGAAAGCCCC
TCAGAACTGGGAAGGCCAGGTAACTCTAGTTACACAGAAACTGTGACT
AAAGTCTATGAAACTGATTACAACAGACTGTAAGAATCAAAGTCAACT
GACATCTATGCTACATATTATTATATAGTTTGTACTGAGCTATTGAAG
TCCCATTAACTTAAAGTATATGTTTTCAAATTGCCATTGCTACTATTG
CTTGTCGGTGTTATTTTATTTTATTGTTTTTGACTTTGGAAGAGATGA
ACTGTGTATTTAACTTAAGCTATTGCTCTTAAAACCAGGGAGTCAGAA
TATATTTGTAAGTTAAATCATTGGTGCTAATAATAAATGTGGATTTTG
TATTAAAATATATAGAAGCAATTTCTGTTTACATGTCCTTGCTACTTT
TAAAAACTTGCATTTATTCCTCAGATTTTAAAAATAAATAAATAATTC
ATTTAAGATTC

IL11 is also known in the art as “interleukin 11”, AGIF, and IL-11. The amino acid sequence of two human IL11 isoforms are:

IL11 (isoform 1: NP_000632.1)
(SEQ ID NO: 3)
MNCVCRLVLVVLSLWPDTAVAPGPPPGPPRVSPDPRAELDSTVLLTRS
LLADTRQLAAQLRDKFPADGDHNLDSLPTLAMSAGALGALQLPGVLTR
LRADLLSYLRHVQWLRRAGGSSLKTLEPELGTLQARLDRLLRRLQLLM
SRLALPQPPPDPPAPPLAPPSSAWGGIRAAHAILGGLHLTLDWAVRGL
LLLKTRL
IL11 (isoform 2: NP_001254647.1)
(SEQ ID NO: 4)
MSAGALGALQLPGVLTRLRADLLSYLRHVQWLRRAGGSSLKTLEPELG
TLQARLDRLLRRLQLLMSRLALPQPPPDPPAPPLAPPSSAWGGIRAAH
AILGGLHLTLDWAVRGLLLLKTRL

A human mRNA encoding the IL11 isoform 1 above is:

IL11 (encodes isoform 1: NM_000641.3)
(presented in DNA form here):
(SEQ ID NO: 5)
ACTGCCGCGGCCCTGCTGCTCAGGGCACATGCCTCCCCTCCCCAGGCC
GCGGCCCAGCTGACCCTCGGGGCTCCCCCGGCAGCGGACAGGGAAGGG
TTAAAGGCCCCCGGCTCCCTGCCCCCTGCCCTGGGGAACCCCTGGCCC
TGTGGGGACATGAACTGTGTTTGCCGCCTGGTCCTGGTCGTGCTGAGC
CTGTGGCCAGATACAGCTGTCGCCCCTGGGCCACCACCTGGCCCCCCT
CGAGTTTCCCCAGACCCTCGGGCCGAGCTGGACAGCACCGTGCTCCTG
ACCCGCTCTCTCCTGGCGGACACGCGGCAGCTGGCTGCACAGCTGAGG
GACAAATTCCCAGCTGACGGGGACCACAACCTGGATTCCCTGCCCACC
CTGGCCATGAGTGCGGGGGCACTGGGAGCTCTACAGCTCCCAGGTGTG
CTGACAAGGCTGCGAGCGGACCTACTGTCCTACCTGCGGCACGTGCAG
TGGCTGCGCCGGGCAGGTGGCTCTTCCCTGAAGACCCTGGAGCCCGAG
CTGGGCACCCTGCAGGCCCGACTGGACCGGCTGCTGCGCCGGCTGCAG
CTCCTGATGTCCCGCCTGGCCCTGCCCCAGCCACCCCCGGACCCGCCG
GCGCCCCCGCTGGCGCCCCCCTCCTCAGCCTGGGGGGGCATCAGGGCC
GCCCACGCCATCCTGGGGGGGCTGCACCTGACACTTGACTGGGCCGTG
AGGGGACTGCTGCTGCTGAAGACTCGGCTGTGACCCGGGGCCCAAAGC
CACCACCGTCCTTCCAAAGCCAGATCTTATTTATTTATTTATTTCAGT
ACTGGGGGCGAAACAGCCAGGTGATCCCCCCGCCATTATCTCCCCCTA
GTTAGAGACAGTCCTTCCGTGAGGCCTGGGGGGCATCTGTGCCTTATT
TATACTTATTTATTTCAGGAGCAGGGGTGGGAGGCAGGTGGACTCCTG
GGTCCCCGAGGAGGAGGGGACTGGGGTCCCGGATTCTTGGGTCTCCAA
GAAGTCTGTCCACAGACTTCTGCCCTGGCTCTTCCCCATCTAGGCCTG
GGCAGGAACATATATTATTTATTTAAGCAATTACTTTTCATGTTGGGG
TGGGGACGGAGGGGAAAGGGAAGCCTGGGTTTTTGTACAAAAATGTGA
GAAACCTTTGTGAGACAGAGAACAGGGAATTAAATGTGTCATACATAT
CCACTTGAGGGCGATTTGTCTGAGAGCTGGGGCTGGATGCTTGGGTAA
CTGGGGCAGGGCAGGTGGAGGGGAGACCTCCATTCAGGTGGAGGTCCC
GAGTGGGCGGGGCAGCGACTGGGAGATGGGTCGGTCACCCAGACAGCT
CTGTGGAGGCAGGGTCTGAGCCTTGCCTGGGGCCCCGCACTGCATAGG
GCCTTTTGTTTGTTTTTTGAGATGGAGTCTCGCTCTGTTGCCTAGGCT
GGAGTGCAGTGAGGCAATCTGAGGTCACTGCAACCTCCACCTCCCGGG
TTCAAGCAATTCTCCTGCCTCAGCCTCCCGATTAGCTGGGATCACAGG
TGTGCACCACCATGCCCAGCTAATTATTTATTTCTTTTGTATTTTTAG
TAGAGACAGGGTTTCACCATGTTGGCCAGGCTGGTTTCGAACTCCTGA
CCTCAGGTGATCCTCCTGCCTCGGCCTCCCAAAGTGCTGGGATTACAG
GTGTGAGCCACCACACCTGACCCATAGGTCTTCAATAAATATTTAATG
GAAGGTTCCACAAGTCACCCTGTGATCAACAGTACCCGTATGGGACAA
AGCTGCAAGGTCAAGATGGTTCATTATGGCTGTGTTCACCATAGCAAA
CTGGAAACAATCTAGATATCCAACAGTGAGGGTTAAGCAACATGGTGC
ATCTGTGGATAGAACGCCACCCAGCCGCCCGGAGCAGGGACTGTCATT
CAGGGAGGCTAAGGAGAGAGGCTTGCTTGGGATATAGAAAGATATCCT
GACATTGGCCAGGCATGGTGGCTCACGCCTGTAATCCTGGCACTTTGG
GAGGACGAAGCGAGTGGATCACTGAAGTCCAAGAGTTCGAGACCGGCC
TGCGAGACATGGCAAAACCCTGTCTCAAAAAAGAAAGAATGATGTCCT
GACATGAAACAGCAGGCTACAAAACCACTGCATGCTGTGATCCCAATT
TTGTGTTTTTCTTTCTATATATGGATTAAAACAAAAATCCTAAAGGGA
AATACGCCAAAATGTTGACAATGACTGTCTCCAGGTCAAAGGAGAGAG
GTGGGATTGTGGGTGACTTTTAATGTGTATGATTGTCTGTATTTTACA
GAATTTCTGCCATGACTGTGTATTTTGCATGACACATTTTAAAAATAA
TAAACACTATTTTTAGAATAACAGAAAAA

A human mRNA encoding the IL11 isoform 2 above is:

IL11 (encodes isoform 2: NM_001267718.1)
(presented in DNA form here):
(SEQ ID NO: 6)
ACTGCCGCGGCCCTGCTGCTCAGGGCACATGCCTCCCCTCCCCAGGCC
GCGGCCCAGCTGACCCTCGGGGCTCCCCCGGCAGCGGACAGGGAAGGG
TTAAAGGCCCCCGGCTCCCTGCCCCCTGCCCTGGGGAACCCCTGGCCC
TGTGGGGACATGAACTAGGGACAAATTCCCAGCTGACGGGGACCACAA
CCTGGATTCCCTGCCCACCCTGGCCATGAGTGCGGGGGCACTGGGAGC
TCTACAGCTCCCAGGTGTGCTGACAAGGCTGCGAGCGGACCTACTGTC
CTACCTGCGGCACGTGCAGTGGCTGCGCCGGGCAGGTGGCTCTTCCCT
GAAGACCCTGGAGCCCGAGCTGGGCACCCTGCAGGCCCGACTGGACCG
GCTGCTGCGCCGGCTGCAGCTCCTGATGTCCCGCCTGGCCCTGCCCCA
GCCACCCCCGGACCCGCCGGCGCCCCCGCTGGCGCCCCCCTCCTCAGC
CTGGGGGGGCATCAGGGCCGCCCACGCCATCCTGGGGGGGCTGCACCT
GACACTTGACTGGGCCGTGAGGGGACTGCTGCTGCTGAAGACTCGGCT
GTGACCCGGGGCCCAAAGCCACCACCGTCCTTCCAAAGCCAGATCTTA
TTTATTTATTTATTTCAGTACTGGGGGCGAAACAGCCAGGTGATCCCC
CCGCCATTATCTCCCCCTAGTTAGAGACAGTCCTTCCGTGAGGCCTGG
GGGGCATCTGTGCCTTATTTATACTTATTTATTTCAGGAGCAGGGGTG
GGAGGCAGGTGGACTCCTGGGTCCCCGAGGAGGAGGGGACTGGGGTCC
CGGATTCTTGGGTCTCCAAGAAGTCTGTCCACAGACTTCTGCCCTGGC
TCTTCCCCATCTAGGCCTGGGCAGGAACATATATTATTTATTTAAGCA
ATTACTTTTCATGTTGGGGTGGGGACGGAGGGGAAAGGGAAGCCTGGG
TTTTTGTACAAAAATGTGAGAAACCTTTGTGAGACAGAGAACAGGGAA
TTAAATGTGTCATACATATCCACTTGAGGGCGATTTGTCTGAGAGCTG
GGGCTGGATGCTTGGGTAACTGGGGCAGGGCAGGTGGAGGGGAGACCT
CCATTCAGGTGGAGGTCCCGAGTGGGCGGGGCAGCGACTGGGAGATGG
GTCGGTCACCCAGACAGCTCTGTGGAGGCAGGGTCTGAGCCTTGCCTG
GGGCCCCGCACTGCATAGGGCCTTTTGTTTGTTTTTTGAGATGGAGTC
TCGCTCTGTTGCCTAGGCTGGAGTGCAGTGAGGCAATCTGAGGTCACT
GCAACCTCCACCTCCCGGGTTCAAGCAATTCTCCTGCCTCAGCCTCCC
GATTAGCTGGGATCACAGGTGTGCACCACCATGCCCAGCTAATTATTT
ATTTCTTTTGTATTTTTAGTAGAGACAGGGTTTCACCATGTTGGCCAG
GCTGGTTTCGAACTCCTGACCTCAGGTGATCCTCCTGCCTCGGCCTCC
CAAAGTGCTGGGATTACAGGTGTGAGCCACCACACCTGACCCATAGGT
CTTCAATAAATATTTAATGGAAGGTTCCACAAGTCACCCTGTGATCAA
CAGTACCCGTATGGGACAAAGCTGCAAGGTCAAGATGGTTCATTATGG
CTGTGTTCACCATAGCAAACTGGAAACAATCTAGATATCCAACAGTGA
GGGTTAAGCAACATGGTGCATCTGTGGATAGAACGCCACCCAGCCGCC
CGGAGCAGGGACTGTCATTCAGGGAGGCTAAGGAGAGAGGCTTGCTTG
GGATATAGAAAGATATCCTGACATTGGCCAGGCATGGTGGCTCACGCC
TGTAATCCTGGCACTTTGGGAGGACGAAGCGAGTGGATCACTGAAGTC
CAAGAGTTCGAGACCGGCCTGCGAGACATGGCAAAACCCTGTCTCAAA
AAAGAAAGAATGATGTCCTGACATGAAACAGCAGGCTACAAAACCACT
GCATGCTGTGATCCCAATTTTGTGTTTTTCTTTCTATATATGGATTAA
AACAAAAATCCTAAAGGGAAATACGCCAAAATGTTGACAATGACTGTC
TCCAGGTCAAAGGAGAGAGGTGGGATTGTGGGTGACTTTTAATGTGTA
TGATTGTCTGTATTTTACAGAATTTCTGCCATGACTGTGTATTTTGCA
TGACACATTTTAAAAATAATAAACACTATTTTTAGAATAACAGAAAAA

### Expression Levels

With regard to the term “expression level” of a an expression product (e.g., an RNA, an mRNA, a protein, etc.), the act of measuring will produce a value referred to herein as an expression level, which represents the amount of the expression product (e.g, RGS1, IL11) measured in the sample. Thus, the term expression product is the molecule being measured (e.g., an RNA encoding RGS1, an RNA encoding IL11, an RGS1 protein, an IL11 protein), and the expression level is a value that represents the amount of the expression product present in the sample (e.g., concentration of protein, number of RNA transcripts, etc.).

An expression level (i.e., level of expression) can be a raw measured value, or can be a normalized and/or weighted value derived from the raw measured value. The terms “expression level” and “measured expression level” are used herein to encompass raw measured values as well as values that have manipulated in some way (e.g., normalized and/or weighted). In some cases, a normalized expression level is a measured expression level of an expression product from a sample where the raw measured value for the expression product has been normalized. For example, the expression level of an expression product (e.g., an RNA encoding RGS1, an RNA encoding IL11, an RGS1 protein, an IL11 protein) can be compared to the expression level of one or more other expression products (e.g., the expression level of a housekeeping gene, the averaged expression levels of multiple genes, etc.) to derive a normalized value that represents a normalized expression level. Methods of normalization will be known to one of ordinary skill in the art and any convenient normalization method can be used. The specific metric (or units) chosen is not crucial as long as the same units are used (or conversion to the same units is performed) when evaluating multiple markers and/or multiple biological samples (e.g., samples from multiple individuals or multiple samples from the same individual).

The expression levels of RGS1 and IL11 (i.e., an expression product of RGS1 and an expression product of IL11) can be measured and utilized in the subject methods. For both RGS1 and IL11, an elevated expression level is associated with being non-responsive to treatment with a TNF inhibitor. In other words, individuals who are non-responsive to treatment with a TNF inhibitor have elevated expression levels of RGS1 and/or IL11 (e.g., an elevated geometric mean of the expression levels of RGS1 and IL11) relative to individuals who are responsive to such treatment. Individuals who are responsive to treatment with a TNF inhibitor have reduced expression levels of RGS1 and/or IL11 (e.g., a reduced geometric mean of the expression levels of RGS1 and IL11) relative to individuals who are non-responsive to such treatment. In yet other words, the expression levels RGS1 and IL11 correlate positively with being non-responsive to treatment with a TNF inhibitor.

In some cases, the expression level (e.g., the number of transcripts, the concentration in a sample, and the like) of an RGS1 expression product (e.g., RNA, protein) in a biological sample from an individual who is non-responsive to treatment with a TNF inhibitor is greater than a reference value (e.g., an expression level of an RGS1 expression product in one or more biological samples from one or more individuals who are responsive to the treatment; a value, e.g., an average, derived from the expression level of an RGS1 expression product in a biological sample from multiple individuals who are responsive to the treatment; etc.). For example, the expression level (e.g., the number of transcripts, the concentration in a sample, and the like) of an RGS1 expression product (e.g., RNA, protein) in a biological sample from an individual who is non-responsive to treatment with a TNF inhibitor can be 1.1-fold or more (e.g., 1.2-fold or more, 1.3-fold or more, 1.4-fold or more, 1.5-fold or more, 2-fold or more, 2.5-fold or more, 3-fold or more, 4-fold or more, 5-fold or more, 7.5-fold or more, or 10-fold or more) greater than a reference value (e.g., an expression level of an RGS1 expression product in one or more biological samples from one or more individuals who are responsive to the treatment; a value, e.g., an average, derived from the expression level of an RGS1 expression product in a biological sample from multiple individuals who are responsive to the treatment; etc.).

In some cases, the expression level (e.g., the number of transcripts, the concentration in a sample, and the like) of an IL11 expression product (e.g., RNA, protein) in a biological sample from an individual who is non-responsive to treatment with a TNF inhibitor is greater than a reference value (e.g., an expression level of an IL11 expression product in one or more biological samples from one or more individuals who are responsive to the treatment; a value, e.g., an average, derived from the expression level of an IL11 expression product in a biological sample from multiple individuals who are responsive to the treatment; etc.). For example, the expression level (e.g., the number of transcripts, the concentration in a sample, and the like) of an IL11 expression product (e.g., RNA, protein) in a biological sample from an individual who is non-responsive to treatment with a TNF inhibitor can be 1.1-fold or more (e.g., 1.2-fold or more, 1.3-fold or more, 1.4-fold or more, 1.5-fold or more, 2-fold or more, 2.5-fold or more, 3-fold or more, 4-fold or more, 5-fold or more, 7.5-fold or more, or 10-fold or more) greater than a reference value (e.g., an expression level of an IL11 expression product in one or more biological samples from one or more individuals who are responsive to the treatment; a value, e.g., an average, derived from the expression level of an IL11 expression product in a biological sample from multiple individuals who are responsive to the treatment; etc.).

In some cases, the combined expression levels (e.g., the geometric mean of the expression levels) of an RGS1 expression product (e.g., RNA, protein) and an IL11 expression product (e.g., RNA, protein) in a biological sample from an individual who is non-responsive to treatment with a TNF inhibitor is greater than a reference value (e.g., combined expression levels, e.g., the geometric mean of the expression levels, of RGS1 and IL11 expression products in one or more biological samples from one or more individuals who are responsive to the treatment; etc.). For example, combined expression levels (e.g., the geometric mean of the expression levels) of an RGS1 expression product (e.g., RNA, protein) and an IL11 expression product (e.g., RNA, protein) in a biological sample from an individual who is non-responsive to treatment with a TNF inhibitor can be 1.1-fold or more (e.g., 1.2-fold or more, 1.3-fold or more, 1.4-fold or more, 1.5-fold or more, 2-fold or more, 2.5-fold or more, 3-fold or more, 4-fold or more, 5-fold or more, 7.5-fold or more, or 10-fold or more) greater than a reference value (e.g., combined expression levels, e.g., the geometric mean of the expression levels, of RGS1 and IL11 expression products in one or more biological samples from one or more individuals who are responsive to the treatment; etc.).

In some cases, the expression level (e.g., the number of transcripts, the concentration in a sample, and the like) of an RGS1 expression product (e.g., RNA, protein) in a biological sample from an individual who is responsive to treatment with a TNF inhibitor is less than a reference value (e.g., an expression level of an RGS1 expression product in one or more biological samples from one or more individuals who are non-responsive to the treatment; a value, e.g., an average, derived from the expression level of an RGS1 expression product in a biological sample from multiple individuals who are non-responsive to the treatment; etc.).

For example, the expression level (e.g., the number of transcripts, the concentration in a sample, and the like) of an RGS1 expression product (e.g., RNA, protein) in a biological sample from an individual who is responsive to treatment with a TNF inhibitor can be reduced by 10% or more (e.g., 20% or more, 30% or more, 40% or more, 50% or more, 60% or more, 70% or more, 80% or more, or 90% or more) compared to a reference value (e.g., an expression level of an RGS1 expression product in one or more biological samples from one or more individuals who are non-responsive to the treatment; a value, e.g., an average, derived from the expression level of an RGS1 expression product in a biological sample from multiple individuals who are non-responsive to the treatment; etc.)

As another example, in some cases, the expression level (e.g., the number of transcripts, the concentration in a sample, and the like) of an RGS1 expression product (e.g., RNA, protein) in a biological sample from an individual who is responsive to treatment with a TNF inhibitor is 95% of a reference value or less (e.g., 90% of the reference value or less, 85% of the reference value or less, 80% of the reference value or less, 75% of the reference value or less, 70% of the reference value or less, 65% of the reference value or less, 60% of the reference value or less, 55% of the reference value or less, 50% of the reference value or less, 45% of the reference value or less, 40% of the reference value or less, 35% of the reference value or less, 30% of the reference value or less, 25% of the reference value or less, 20% of the reference value or less, 15% of the reference value or less, 10% of the reference value or less, or 5% of the reference value or less). Examples of a suitable reference value in such cases include but are not limited to: an expression level of an RGS1 expression product in one or more biological samples from one or more individuals who are non-responsive to treatment with a TNF inhibitor; a value (e.g., an average, a mean, a median, a geometric mean, etc.) derived from the expression level of an RGS1 expression product in a biological sample from multiple individuals who are non-responsive to treatment with a TNF inhibitor; etc.)

In some cases, the expression level (e.g., the number of transcripts, the concentration in a sample, and the like) of an IL11 expression product (e.g., RNA, protein) in a biological sample from an individual who is responsive to treatment with a TNF inhibitor is less than a reference value (e.g., an expression level of an IL11 expression product in one or more biological samples from one or more individuals who are non-responsive to the treatment; a value, e.g., an average, derived from the expression level of an IL11 expression product in a biological sample from multiple individuals who are non-responsive to the treatment; etc.).

For example, the expression level (e.g., the number of transcripts, the concentration in a sample, and the like) of an IL11 expression product (e.g., RNA, protein) in a biological sample from an individual who is responsive to treatment with a TNF inhibitor can be reduced by 10% or more (e.g., 20% or more, 30% or more, 40% or more, 50% or more, 60% or more, 70% or more, 80% or more, or 90% or more) compared to a reference value (e.g., an expression level of an IL11 expression product in one or more biological samples from one or more individuals who are non-responsive to the treatment; a value, e.g., an average, derived from the expression level of an IL11 expression product in a biological sample from multiple individuals who are non-responsive to the treatment; etc.)

As another example, in some cases, the expression level (e.g., the number of transcripts, the concentration in a sample, and the like) of an IL11 expression product (e.g., RNA, protein) in a biological sample from an individual who is responsive to treatment with a TNF inhibitor is 95% of a reference value or less (e.g., 90% of the reference value or less, 85% of the reference value or less, 80% of the reference value or less, 75% of the reference value or less, 70% of the reference value or less, 65% of the reference value or less, 60% of the reference value or less, 55% of the reference value or less, 50% of the reference value or less, 45% of the reference value or less, 40% of the reference value or less, 35% of the reference value or less, 30% of the reference value or less, 25% of the reference value or less, 20% of the reference value or less, 15% of the reference value or less, 10% of the reference value or less, or 5% of the reference value or less). Examples of a suitable reference value in such cases include but are not limited to: an expression level of an IL11 expression product in one or more biological samples from one or more individuals who are non-responsive to treatment with a TNF inhibitor; a value (e.g., an average, a mean, a median, a geometric mean, etc.) derived from the expression level of an IL11 expression product in a biological sample from multiple individuals who are non-responsive to treatment with a TNF inhibitor; etc.).

In some cases, the combined expression levels (e.g., the geometric mean of the expression levels) of an RGS1 expression product (e.g., RNA, protein) and an IL11 expression product (e.g., RNA, protein) in a biological sample from an individual who is responsive to treatment with a TNF inhibitor is less than a reference value (e.g., combined expression levels, e.g., the geometric mean of the expression levels, of RGS1 and IL11 expression products in one or more biological samples from one or more individuals who are non-responsive to the treatment; etc.).

For example, combined expression levels (e.g., the geometric mean of the expression levels) of an RGS1 expression product (e.g., RNA, protein) and an IL11 expression product (e.g., RNA, protein) in a biological sample from an individual who is responsive to treatment with a TNF inhibitor can be reduced by 10% or more (e.g., 20% or more, 30% or more, 40% or more, 50% or more, 60% or more, 70% or more, 80% or more, or 90% or more) compared to a reference value (e.g., combined expression levels, e.g., the geometric mean of the expression levels, of RGS1 and IL11 expression products in one or more biological samples from one or more individuals who are non-responsive to the treatment; etc.).

As another example, in some cases, the combined expression levels (e.g., the geometric mean of the expression levels) of an RGS1 expression product (e.g., RNA, protein) and an IL11 expression product (e.g., RNA, protein) in a biological sample from an individual who is responsive to treatment with a TNF inhibitor is 95% of a reference value or less (e.g., 90% of the reference value or less, 85% of the reference value or less, 80% of the reference value or less, 75% of the reference value or less, 70% of the reference value or less, 65% of the reference value or less, 60% of the reference value or less, 55% of the reference value or less, 50% of the reference value or less, 45% of the reference value or less, 40% of the reference value or less, 35% of the reference value or less, 30% of the reference value or less, 25% of the reference value or less, 20% of the reference value or less, 15% of the reference value or less, 10% of the reference value or less, or 5% of the reference value or less). An example of a suitable reference value in such cases includes but is not limited to: a value representing the combined expression levels (e.g., the geometric mean of the expression levels) of RGS1 and IL11 expression products in one or more biological samples from one or more individuals who are non-responsive to treatment with a TNF inhibitor.

### Measuring Expression Levels

The terms “assaying” and “measuring” are used herein to include the physical steps of manipulating a biological sample to generate data related to a sample (e.g., measuring an expression level in a biological sample). As will be readily understood by one of ordinary skill in the art, a biological sample can be “obtained” prior to assaying the sample. The terms “obtained” or “obtaining” as used herein encompass the physical extraction or isolation of a biological sample from a subject. The terms “obtained” or “obtaining” as used herein also encompasses the act of receiving an extracted or isolated biological sample. For example, a testing facility can “obtain” a biological sample in the mail (or via delivery, etc.) prior to assaying the sample. In some such cases, the biological sample was “extracted” or “isolated” (and thus “obtained”) from the subject by a second entity prior to mailing, and then “obtained” by the testing facility upon arrival of the sample. Thus, the testing facility can obtain the sample and then assay the sample (e.g., measure expression levels from the sample), thereby producing data related to the sample. Alternatively, a biological sample can be extracted or isolated from a subject by the same person or same entity that subsequently assays the sample. In some embodiments, a subject method includes: obtaining a biological sample and measuring the expressional level of an RGS1 expression product and/or an expression level of an IL11 expression product in the sample.

In practicing the subject methods, the expression level of an RGS1 expression product (e.g., mRNA, protein) and/or the expression level of an IL11 expression product (e.g., mRNA, protein) in a biological sample from an individual can be measured. The expression level(s) can be measured by any convenient method. For example, RNA expression levels can be detected by measuring the levels/amounts of one or more nucleic acid transcripts, e.g. mRNAs, of the specified gene (e.g., RGS1 and/or IL11). Protein expression levels (e.g., RGS1 and/or IL11) can be detected by measuring the levels/amounts of the RGS1 and/or IL11 protein(s).

The terms “measuring” and “analyzing” are used herein to refer to any form of measurement, and include determining if an element is present or not. These terms include both quantitative and/or qualitative determinations. Assaying may be relative or absolute. For example, “measuring” can be used to determine whether the measured expression level is less than, great than, “less than or equal to”, or “greater than or equal to” a particular threshold, (the threshold can be pre-determined or can be determined by assaying a control sample). On the other hand, “measuring to determine the expression level” or simply “measuring expression levels” can mean determining a quantitative value (using any convenient metric) that represents the level of expression (i.e., expression level, e.g., the amount of protein and/or RNA, e.g., mRNA) of a particular biomarker (e.g., RGS1 mRNA, IL11 mRNA, RGS1 protein, and/or IL11 protein). The level of expression can be expressed in arbitrary units associated with a particular assay (e.g., fluorescence units, e.g., mean fluorescence intensity (MFI), threshold cycle (Cₜ), quantification cycle (C_{q}), and the like), or can be expressed as an absolute value with defined units (e.g., number of mRNA transcripts, number of protein molecules, concentration of protein, etc.).

The markers used herein (e.g., RGS1 and/or IL11) may include proteins and/or their corresponding genetic sequences, i.e. mRNA, DNA, etc. By a “gene” or “recombinant gene” it is meant a nucleic acid comprising an open reading frame that encodes for the protein. The boundaries of a coding sequence are determined by a start codon at the 5′ (amino) terminus and a translation stop codon at the 3′ (carboxy) terminus. A transcription termination sequence may be located 3′ to the coding sequence. In addition, a gene may optionally include its natural promoter (i.e., the promoter with which the exons and introns of the gene are operably linked in a non-recombinant cell, i.e., a naturally occurring cell), and associated regulatory sequences, and may or may not have sequences upstream of the AUG start site (e.g., 5′ UTR), and may or may not include untranslated leader sequences, signal sequences, downstream untranslated sequences (e.g., 3′ UTR), transcriptional start and stop sequences, polyadenylation signals, translational start and stop sequences, ribosome binding sites, and the like. When referring to an a marker herein, it is meant any nucleic acid and/or amino acid sequence that can be identified that is uniquely associated with the corresponding gene. For example, if the 5′UTR of RGS1 and/or IL11 contains a first sequence that is unique to that particular gene, then the associated biomarker can be a sequence that includes that first unique sequence.

### Measuring RNA

An expression level of an expression product (e.g., an expression product of RGS1 and/or IL11) may be measured by detecting in a patient sample (a biological sample from an individual, e.g., an individual with any of the diseases or disorders described above, e.g., IBD) the amount or level of one or more RNA transcripts or a fragment thereof encoded by the gene of interest. For measuring RNA levels, the amount or level of an RNA in the sample is determined, e.g., the expression level of an mRNA. In some instances, the expression level of one or more additional RNAs may also be measured, and the level of biomarker expression compared to the level of the one or more additional RNAs to provide a normalized value for the biomarker expression level.

The expression level of nucleic acids in the sample may be detected using any convenient protocol. A number of exemplary methods for measuring RNA (e.g., mRNA) expression levels (e.g., expression level of a nucleic acid biomarker) in a sample are known by one of ordinary skill in the art, such as those methods employed in the field of differential gene expression analysis, and any convenient method can be used. Exemplary methods include, but are not limited to: hybridization-based methods (e.g., Northern blotting, array hybridization (e.g., microarray); in situ hybridization; in situ hybridization followed by FACS; and the like)(Parker & Barnes, Methods in Molecular Biology 106:247-283 (1999)); RNAse protection assays (Hod, Biotechniques 13:852-854 (1992)); PCR-based methods (e.g., reverse transcription PCR (RT-PCR), quantitative RT-PCR (qRT-PCR), real-time RT-PCR, etc.)(Weis et al., Trends in Genetics 8:263-264 (1992)); nucleic acid sequencing methods (e.g., Sanger sequencing, Next Generation sequencing (i.e., massive parallel high throughput sequencing, e.g., Illumina's reversible terminator method, Roche's pyrosequencing method (454), Life Technologies' sequencing by ligation (the SOLiD platform), Life Technologies' Ion Torrent platform, single molecule sequencing, etc.); nanopore based sequencing methods; and the like.

In some embodiments, the biological sample can be assayed directly. In some embodiments, nucleic acid of the biological sample is amplified (e.g., by PCR) prior to assaying. As such, techniques such as PCR (Polymerase Chain Reaction), RT-PCR (reverse transcriptase PCR), qRT-PCR (quantitative RT-PCR, real time RT-PCR), etc. can be used prior to the hybridization methods and/or the sequencing methods discussed above.

As noted above, gene expression in a sample can be detected using hybridization analysis, which is based on the specificity of nucleotide interactions. Oligonucleotides or cDNA can be used to selectively identify or capture DNA or RNA of specific sequence composition, and the amount of RNA or cDNA hybridized to a known capture sequence determined qualitatively or quantitatively, to provide information about the relative representation of a particular message within the pool of cellular messages in a sample. Hybridization analysis can be designed to allow for concurrent screening of the relative expression of hundreds to thousands of genes by using, for example, array-based technologies having high density formats, including filters, microscope slides, or microchips, or solution-based technologies that use spectroscopic analysis.

Hybridization to arrays may be performed, where the arrays can be produced according to any suitable methods known in the art. For example, methods of producing large arrays of oligonucleotides are described in U.S. Pat. No. 5,134,854, and U.S. Pat. No. 5,445,934 using light-directed synthesis techniques. Using a computer controlled system, a heterogeneous array of monomers is converted, through simultaneous coupling at a number of reaction sites, into a heterogeneous array of polymers. Alternatively, microarrays are generated by deposition of pre-synthesized oligonucleotides onto a solid substrate, for example as described in PCT published application no. WO 95/35505.

Methods for collection of data from hybridization of samples with an array are also well known in the art. For example, the polynucleotides of the cell samples can be generated using a detectable fluorescent label, and hybridization of the polynucleotides in the samples detected by scanning the microarrays for the presence of the detectable label. Methods and devices for detecting fluorescently marked targets on devices are known in the art. Generally, such detection devices include a microscope and light source for directing light at a substrate. A photon counter detects fluorescence from the substrate, while an x-y translation stage varies the location of the substrate. A confocal detection device that can be used in the subject methods is described in U.S. Pat. No. 5,631,734. A scanning laser microscope is described in Shalon et al., Genome Res. (1996) 6:639. A scan, using the appropriate excitation line, is performed for each fluorophore used. The digital images generated from the scan are then combined for subsequent analysis. For any particular array element, the ratio of the fluorescent signal from one sample is compared to the fluorescent signal from another sample, and the relative signal intensity determined.

Methods for analyzing the data collected from hybridization to arrays are well known in the art. For example, where detection of hybridization involves a fluorescent label, data analysis can include the steps of determining fluorescent intensity as a function of substrate position from the data collected, removing outliers, i.e. data deviating from a predetermined statistical distribution, and calculating the relative binding affinity of the targets from the remaining data. The resulting data can be displayed as an image with the intensity in each region varying according to the binding affinity between targets and probes.

One representative and convenient type of protocol for measuring mRNA levels is array-based gene expression profiling. Such protocols are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively.

Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the phenotype determinative genes whose expression is being assayed is contacted with target nucleic acids as described above. Contact is carried out under hybridization conditions, e.g., stringent hybridization conditions, and unbound nucleic acid is then removed. The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

The resultant pattern of hybridized nucleic acid provides information regarding expression for each of the genes that have been probed, where the expression information is in terms of whether or not the gene is expressed and, typically, at what level, where the expression data, i.e., expression profile (e.g., in the form of a transcriptosome), may be both qualitative and quantitative. Pattern analysis can be performed manually, or can be performed using a computer program. Methods for preparation of substrate matrices (e.g., arrays), design of oligonucleotides for use with such matrices, labeling of probes, hybridization conditions, scanning of hybridized matrices, and analysis of patterns generated, including comparison analysis, are described in, for example, U.S. Pat. No. 5,800,992.

Alternatively, non-array based methods for quantitating the level of one or more nucleic acids in a sample may be employed. These include those based on amplification protocols, e.g., Polymerase Chain Reaction (PCR)-based assays, including quantitative PCR, reverse-transcription PCR (RT-PCR), real-time PCR, quantitative RT-PCR (qRT-PCR), and the like, e.g. TaqMan® RT-PCR, SYBR green; MassARRAY® System, BeadArray® technology, and Luminex technology; and those that rely upon hybridization of probes to filters, e.g. Northern blotting and in situ hybridization. Other non-amplified methods of analysis include digital bar-coding, e.g. NanoString nCounter Analysis System which is a digital color-coded barcode technolog based on direct multiplexed measurement of gene expression. The technology uses molecular “barcodes” and single molecule imaging to detect and count hundreds of unique transcripts in a single reaction. Each color-coded barcode is attached to a single target-specific probe corresponding to a gene of interest. Mixed together with controls, they form a multiplexed CodeSet.

Examples of some of the nucleic acid sequencing methods listed above are described in the following references: Margulies et al (Nature 2005 437: 376-80); Ronaghi et al (Analytical Biochemistry 1996 242: 84-9); Shendure (Science 2005 309: 1728); Imelfort et al (Brief Bioinform. 2009 10:609-18); Fox et al (Methods Mol Biol. 2009; 553:79-108); Appleby et al (Methods Mol Biol. 2009; 513:19-39); Soni et al Clin Chem 53: 1996-2001 2007; and Morozova (Genomics. 2008 92:255-64), which are incorporated by reference for the general descriptions of the methods and the particular steps of the methods, including starting products, reagents, and final products for each of the steps.

For measuring mRNA levels, the starting material is typically total RNA or poly A+ RNA isolated from a biological sample (e.g., suspension of cells from a peripheral blood sample, an aspirate, a formalin-fixed paraffin embedded (FFPE) tissue sample, a biopsy sample, an FFPE biopsy sample, etc., or from a homogenized tissue, e.g. a homogenized biopsy sample, a homogenized paraffin- or OCT-embedded sample, etc.). General methods for mRNA extraction are known in the art and are disclosed in standard textbooks of molecular biology, including Ausubel et al., Current Protocols of Molecular Biology, John Wiley and Sons (1997). RNA isolation (e.g., mRNA isolation) can be performed using any convenient protocol. For example, RNA isolation can be performed using a purification kit, buffer set and protease from commercial manufacturers, according to the manufacturer's instructions. For example, RNA from cell suspensions can be isolated using Qiagen RNeasy mini-columns, and RNA from cell suspensions or homogenized tissue samples can be isolated using the TRIzol reagent-based kits (Invitrogen), MasterPure™ Complete DNA and RNA Purification Kit (EPICENTRE™, Madison, Wis.), Paraffin Block RNA Isolation Kit (Ambion, Inc.) or RNA Stat-60 kit (Tel-Test).

### Measuring Protein

An expression level of an expression product (e.g., an expression product of RGS1 and/or IL11) may be measured by detecting in a patient sample (a biological sample from an individual, e.g., an individual with any of the diseases or disorders described above, e.g., IBD) the amount or level of one or more proteins (e.g., RGS1 and/or IL11) or a fragment thereof encoded. For measuring protein levels, the amount or level of a polypeptide in the biological sample is determined. In some instances, the concentration of one or more additional proteins may also be measured, and the measured expression level compared to the level of the one or more additional proteins to provide a normalized value for the measured expression level. In some embodiments, the measured expression level is a relative value calculated by comparing the level of one protein relative to another protein. In other embodiments the concentration is an absolute measurement (e.g., weight/volume or weight/weight).

The expression level of a protein (e.g., RGS1 and/or IL11) may be measured by detecting in a sample the amount or level of one or more proteins/polypeptides or fragments thereof to arrive at a protein level representation. The terms “polypeptide,” “peptide” and “protein” are used interchangeably herein to refer to a polymer of amino acid residues. “Polypeptide” refers to a polymer of amino acids (amino acid sequence) and does not refer to a specific length of the molecule. Thus peptides and oligopeptides are included within the definition of polypeptide. This term also refers to or includes post-translationally modified polypeptides, for example, glycosylated polypeptide, acetylated polypeptide, phosphorylated polypeptide and the like. Included within the definition are, for example, polypeptides containing one or more analogs of an amino acid, polypeptides with substituted linkages, as well as other modifications known in the art, both naturally occurring and non-naturally occurring.

In some embodiments, the extracellular protein level is measured. For example, in some cases, the protein (i.e., polypeptide) being measured is a secreted protein (e.g., IL11) and the concentration can therefore be measured in the extracellular fluid of a biological sample (e.g., the concentration of a protein can be measured in the serum, in fluid from a region of inflammation, in an aspirate of the lungs, in fluid surrounding a biopsy, in extracellular fluid from a biopsy, etc.). In some cases, the cells are removed from the biological sample (e.g., via centrifugation, via adhering cells to a dish or to plastic, etc.) prior to measuring the concentration. In some cases, the intracellular protein level is measured by lysing cells of the biological sample (e.g., cells isolated from a region on inflammation, cells from a biopsy, etc.) to measure the level of protein in the cellular contents. In some cases, both the extracellular and cell-associated levels of protein are measured by separating the cellular and fluid portions of the biological sample (e.g., via centrifugation), measuring the extracellular level of the protein by measuring the level of protein in the fluid portion of the biological sample, and measuring the cell-associated level of protein by measuring the level of protein in the cell-associated portion of the biological sample (e.g., after lysing the cells). In some cases, the total level of protein (i.e., combined extracellular and cell-associated protein) is measured by lysing the cells of the biological sample to include the cell-associated protein contents as part of the sample.

When protein levels are to be detected, any convenient protocol for measuring protein levels may be employed. Examples of methods for assaying protein levels include but are not limited to enzyme-linked immunosorbent assay (ELISA), mass spectrometry, proteomic arrays, xMAP™ microsphere technology, flow cytometry, western blotting, immunohistochemistry, and the like.

Some protein detection methods are antibody-based methods. The term “antibody” is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity. “Antibodies” (Abs) and “immunoglobulins” (Igs) are glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lymph system and at increased levels by myelomas. “Antibody fragment”, and all grammatical variants thereof, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules (2) single chain polypeptides containing only one light chain variable domain, or a fragment thereof that contains the three CDRs of the light chain variable domain, without an associated heavy chain moiety (3) single chain polypeptides containing only one heavy chain variable region, or a fragment thereof containing the three CDRs of the heavy chain variable region, without an associated light chain moiety and (4) nanobodies comprising single Ig domains from non-human species or other specific single-domain binding modules; and multispecific or multivalent structures formed from antibody fragments. In an antibody fragment comprising one or more heavy chains, the heavy chain(s) can contain any constant domain sequence (e.g. CH1 in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s).

As used in this disclosure, the term “epitope” means any antigenic determinant on an antigen to which the paratope of an antibody binds. Epitopic determinants usually consist of chemically active surface groupings of molecules such as amino acids or sugar side chains and usually have specific three dimensional structural characteristics, as well as specific charge characteristics.

The terms “specific binding,” “specifically binds,” and the like, refer to non-covalent or covalent preferential binding to a molecule relative to other molecules or moieties in a solution or reaction mixture (e.g., an antibody specifically binds to a particular polypeptide or epitope relative to other available polypeptides). In some embodiments, the affinity of one molecule for another molecule to which it specifically binds is characterized by a K_{D} (dissociation constant) of 10⁻⁵ M or less (e.g., 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, or 10⁻¹⁶ M or less). “Affinity” refers to the strength of binding, increased binding affinity being correlated with a lower K_{D}.

The term “specific binding member” as used herein refers to a member of a specific binding pair (i.e., two molecules, usually two different molecules, where one of the molecules, e.g., a first specific binding member, through non-covalent means specifically binds to the other molecule, e.g., a second specific binding member).

The term “specific binding agent” as used herein refers to any agent that specifically binds a biomolecule (e.g., a marker such as a nucleic acid marker molecule, a protein marker molecule, etc.). In some cases, a “specific binding agent” for a marker molecule (e.g., a biomarker) is used. Specific binding agents can be any type of molecule. In some cases, a specific binding agent is an antibody or a fragment thereof. In some cases, a specific binding agent is nucleic acid probe (e.g., an RNA probe; a DNA probe; an RNA/DNA probe; a modified nucleic acid probe, e.g., a locked nucleic acid (LNA) probe, a morpholino probe, etc.; and the like).

### Calculating a TNF Inhibitor Score

Aspects of the disclosure include obtaining (e.g., via calculating) a TNF inhibitor signature score for an individual. Once a value for the expression level of an RGS1 expression product (e.g., RNA, protein) of and an IL11 expression product (e.g., RNA, protein) has been obtained (via measuring), the measurement(s) may be analyzed in a number of ways to obtain a TNF inhibitor score. By a “TNF inhibitor signature score” it is meant a single metric value that represents a combination of measured expression levels (e.g., in some cases normalized and/or weighted expression levels) of an RGS1 expression product and an IL11 expression product. A TNF inhibitor signature score can be arrived at (produced/generated) by calculation from the measured expression levels of the RGS1 and IL11 expression products (e.g., from the raw measured values, from normalized expression levels, from weighted expression levels, from normalized and weighted expression levels, etc.).

A TNF inhibitor signature score for an individual may be calculated by any convenient method and/or algorithm for calculating biomarker scores. For example, weighted marker levels, e.g. log₂ transformed and normalized marker levels that have been weighted by, e.g., multiplying each normalized marker level to a weighting factor, may be totaled and in some cases averaged to arrive at a TNF inhibitor signature score.

In some instances, the weighting factor, or simply “weight” for each marker (e.g., RGS1 RNA, IL11 RNA, RGS1 protein, and/or IL11 protein) in a panel may be a reflection of the change in analyte level in the sample. The weights may be reflective of the importance of each marker to the specificity, sensitivity and/or accuracy of the marker panel in making the diagnostic, prognostic, or monitoring assessment. Such weights may be determined by any convenient method, e.g., statistical machine learning methodology, e.g. Principal Component Analysis (PCA), linear regression, support vector machines (SVMs), and/or random forests of the dataset from which the sample was obtained may be used. In some instances, weights for each marker are defined by the dataset from which the patient sample was obtained. In other instances, weights for each marker may be defined based on a reference dataset, or “training dataset”. Any dataset relating to individuals as responders and/or non-responders to treatment with a TNF inhibitor may be used as a reference dataset. For example, the weights may be determined based upon any of the datasets and/or results provided in the examples section below.

In some cases, calculating a TNF inhibitor signature score includes normalizing and/or weighting the raw measured expression levels. In some cases, calculating a TNF inhibitor signature includes calculating the geometric mean of the measured expression levels (e.g., raw measured values, normalized expression levels, weighted expression levels, normalized and weighted expression levels, etc.) of an RGS1 expression product and an IL11 expression product. A geometric mean is a type of mean or average, which indicates the central tendency or typical value of a set of numbers by using the product of their values (as opposed to the arithmetic mean which uses their sum). The geometric mean is defined as the nth root of the product of n numbers, e.g., the formula (R₁R₂)^{1/2} where R₁ and R₂ are the expression levels for RGS1 and IL11.

The measured expression levels can be log₂ transformed and/or normalized (e.g., relative to the expression of one or more housekeeping genes such as AGPAT1, PRPF40A, ABL1, GAPDH, PGK1, ACTB, RPLPO, GUS, TFRC, HPRT1, ESD, GUSB, HMBS, B2M, IPO8, PPIA, PGK1, RPS11, RPL0, RPL10, RPL14, RPL18, BAT1, TBP, and the like; relative to the signal across a whole panel, e.g., relative to the overall number of “reads” in a sample, etc). The expression levels of RGS1 and/or IL11 expression products can also be weighted.

The resultant data (from measuring the expression levels, RNA and/or protein, of NSCLC markers) provides information regarding levels in the sample for each of the markers that have been probed, wherein the information is in terms of whether or not the marker is present and, typically, at what level, and wherein the data may be qualitative and/or quantitative.

Relative quantification (also called normalization) can be accomplished by comparison of detected levels or amounts between two or more different target analytes to provide a relative quantification of each of the two or more different analytes, e.g., relative to each other. In some cases, normalization can be accomplished by comparison of detected levels of an analyte followed by normalization. For example, in cases where a nucleic acid analyte is quantified by counting (e.g., counting the number of “reads” that map to (i.e., can be assigned to) the analyte of interest when performing high throughput sequencing methods), the number of “reads” and/or “fragments” counted for the target analyte can be normalized to the number of overall reads in the sample and/or can be normalized for the length of the target nucleic acid (this type of normalization typically results in reads per thousand bases per million reads (RPKM) or fragments per thousand bases per million reads (FPKM) as is known in the art). Any convenient means of normalization can be performed. As non-limiting examples, normalization techniques can include: using algorithms such as the MASS algorithm (see, e.g, Pepper et al, BMC Bioinformatics 2007, 8:273), quantile normalization, and/or Robust Multi-array Average (RMA). Any convenient method for normalization can be used and many methods will be available to one of ordinary skill in the art. For example, normalization methods have been developed and will be available for both nucleic acid and protein measurement assays (including, for example, microarray assays, quantitative PCR (qRT-PCR, qPCR) assays, ELISA assays, mass spec based assays, etc.).

In some cases, if an individual's calculated TNF inhibitor signature score is less than a reference value, the individual is a responder to treatment with a TNF inhibitor (e.g., the individual can be predicted to positively respond to treatment with a TNF inhibitor). In some cases, if an individual's calculated TNF inhibitor signature score is less than or equal to a reference value, the individual is a responder to treatment with a TNF inhibitor (e.g., the individual can be predicted to positively respond to treatment with a TNF inhibitor).

In some cases, if an individual's calculated TNF inhibitor signature score is greater than a reference value, the individual is a non-responder to treatment with a TNF inhibitor (e.g., it can be predicted that the individual will not respond positively to treatment with a TNF inhibitor). In some cases, if an individual's calculated TNF inhibitor signature score is greater than or equal to a reference value, the individual is a non-responder to treatment with a TNF inhibitor (e.g., it can be predicted that the individual will not respond positively to treatment with a TNF inhibitor).

These methods of analysis can be readily performed by one of ordinary skill in the art, e.g., by employing a computer-based system, e.g. using any hardware, software and data storage medium as is known in the art, and employing any algorithms convenient for such analysis. For example, data mining algorithms can be applied through “cloud computing”, smartphone based or client-server based platforms, and the like.

In some cases, the subject methods include providing the TNF inhibitor score as a part of a report. Thus, in some instances, the subject methods include a step of generating or outputting a report providing the results of an evaluation (e.g., calculation of a TNF inhibitor score) of an expression level of an RGS1 expression product (e.g., RNA, protein) and an expression level of an IL11 expression product (e.g., RNA, protein) in a sample, which report can be provided in the form of a non-transient electronic medium (e.g., an electronic display on a computer monitor, stored in memory, etc.), or in the form of a tangible medium (e.g., a report printed on paper or other tangible medium). Any form of report may be provided, e.g. as known in the art or as described in greater detail below.

### Treatment

Subject methods and/or reports may include: recommending a treatment regimen (e.g., a therapy)(e.g., a therapy that includes administration of a TNF inhibitor or a therapy that does not include administration of a TNF inhibitor) based on a prognosis (e.g., prediction that the individual is a responder or non-responder to treatment with a TNF inhibitor); prescribing a treatment regimen (e.g., a therapy that includes administration of a TNF inhibitor or a therapy that does not include administration of a TNF inhibitor); and/or administering a treatment (e.g., a therapy that includes administration of a TNF inhibitor or a therapy that does not include administration of a TNF inhibitor). For example, in some cases, a subject method includes a step of treating the individual with a TNF inhibitor or a step of treating the individual with a therapy that does not include administration of a TNF inhibitor.

In some embodiments, a “treatment recommendation” is provided for the individual based on a prognosis (e.g., guidance to a clinician as to a treatment recommendation for the individual based on the prognosis). For example, in some cases, a subject method includes a step of recommending a therapy for an individual.

In some cases, a subject method includes a step of recommending a therapy that includes administration of a TNF inhibitor, or a step of recommending a therapy that does not include administration of a TNF inhibitor. In other words, in some cases, a recommended therapy (treatment) includes administration of a TNF inhibitor and in some cases a recommended therapy does not include administration of a TNF inhibitor. In some cases the provided recommendation is to not administer a therapy that includes a TNF inhibitor.

Various treatments for individuals having an autoimmune and/or an immune-mediated disorder (e.g., inflammatory bowel disease (IBD), psoriasis, plaque psoriasis, psoriatic arthritis, Crohn's disease (CD), ulcerative colitis (UC), rheumatoid arthritis (RA), ankylosing spondylitis, hidradenitis suppurativa, refractory asthma) will be known to one of ordinary skill in the art such as therapies that include administration of a TNF inhibitor as well as therapies that do not include administration of a TNF inhibitor.

The terms “treatment”, “treating”, “treat” and the like are used herein to generally refer to obtaining a desired pharmacologic and/or physiologic effect. The effect can completely or partially prevent progression of a disease or symptom(s) (e.g., an autoimmune and/or an immune-mediated disorder such as inflammatory bowel disease (IBD), psoriasis, plaque psoriasis, psoriatic arthritis, Crohn's disease (CD), ulcerative colitis (UC), rheumatoid arthritis (RA), ankylosing spondylitis, hidradenitis suppurativa, and/or refractory asthma) thereof and/or may be therapeutic in terms of a partial or complete cure for (e.g., reversal of) a disease/disorder and/or adverse effect attributable to the disease/disorder.

The term “treatment” encompasses any treatment of a disease in a mammal, particularly a human, and includes: (a) inhibiting a disease and/or symptom(s), i.e., arresting development (e.g., preventing progression) of a disease and/or the associated symptoms; or (b) relieving the disease and the associated symptom(s), i.e., causing regression of the disease and/or symptom(s). For example, in some cases, treating with a TNF inhibitor inhibits (e.g., prevents the progression of) inflammation (e.g., inflammation associated with a disease). In some cases, treating with a TNF inhibitor reduces (e.g., causes regression of) inflammation (e.g., inflammation associated with a disease). Individuals in need of treatment can include those with an autoimmune disease and/or an immune-mediated disorder such as inflammatory bowel disease (IBD), psoriasis, plaque psoriasis, psoriatic arthritis, Crohn's disease (CD), ulcerative colitis (UC), rheumatoid arthritis (RA), ankylosing spondylitis, hidradenitis suppurativa, and/or refractory asthma.

As such, a responder to treatment with a TNF inhibitor is an individual who upon the administration of a TNF inhibitor, exhibits a desired pharmacologic and/or physiologic effect (e.g., preventing progression of the disease and/or symptom(s), e.g., inflammation, and/or reversal/regression of the disease and/or symptom(s), e.g., inflammation). On the other hand, a non-responder to treatment with a TNF inhibitor is an individual who upon the administration of a TNF inhibitor, does not exhibit a desired pharmacologic and/or physiologic effect (e.g., preventing progression of the disease and/or symptom(s), e.g., inflammation, and/or reversal/regression of the disease and/or symptom(s), e.g., inflammation). The inventors have discovered that prior to administration of a TNF inhibitor, the subject methods can be used to predict whether a given individual is a responder or non-responder to treatment with a TNF inhibitor. In other words, the inventors have discovered that prior to administration of a TNF inhibitor the subject methods can be used to predict whether a given individual will positively respond to the treatment (e.g., predict whether the individual will exhibit the desired pharmacologic and/or physiologic effect after administration of a TNF inhibitor).

### Predicting/Providing a Prognosis

Aspects of the disclosure include a step of predicting that an individual is a responder to treatment with a TNF inhibitor (i.e., predicting that the individual will respond (positively) to treatment with a TNF inhibitor) or predicting that an individual is a non-responder to treatment with a TNF inhibitor (i.e., predicting that the individual will not respond (positively) to treatment with a TNF inhibitor). In some cases, a subject method includes a step of predicting or providing a prediction. By “providing a prognosis” or “providing a prediction” for an individual (e.g. an individual with an autoimmune and/or immune-mediated disorder such as inflammatory bowel disease (IBD), psoriasis, plaque psoriasis, psoriatic arthritis, Crohn's disease (CD), ulcerative colitis (UC), rheumatoid arthritis (RA), ankylosing spondylitis, hidradenitis suppurativa, or refractory asthma,” it is generally meant providing a prediction of the responsiveness of the individual to a therapy that includes administration of a TNF inhibitor (i.e., a prediction as to whether the individual is a responder or a non-responder to treatment with a TNF inhibitor). The predictive methods described herein can be used to assist patients and physicians in making treatment decisions, e.g. in choosing the most appropriate treatment modalities for any particular patient.

In some embodiments, a prediction is based on a comparison with one or more reference values. For example, in some embodiments, an expression level of an RGS1 expression product, an expression level of an IL11 expression product, and/or a TNF inhibitor signature score (e.g., calculated from expression levels of RGS1 and IL11 expression products) from an individual (e.g., calculated and/or measured from a biological sample from the individual) is compared to an expression level of an RGS1 expression product, an expression level of an IL11 expression product, and/or a TNF inhibitor signature score (e.g., calculated from expression levels of RGS1 and IL11 expression products) from a reference individual (e.g., calculated and/or measured from a biological sample from an individual or individual(s) who are known to be a responder(s) or a non-responder(s) to treatment with a TNF inhibitor). In some cases, a reference vale or multiple reference values is/are provided as part of a report.

The terms “reference value” and “control value” as used herein mean a standardized value (e.g., that represents a standardized expression level) to be used to interpret the expression level(s) measured from a test individual. The reference value or control value is typically an expression level or TNF inhibitor signature score that is obtained from a biological sample (e.g., cell/tissue) from an individual (or an average value from multiple individuals) with a known phenotype (e.g., responder or non-responder to treatment with a TNF inhibitor.)

For example, an expression level or TNF inhibitor signature score of a test individual can be compared with a reference value (e.g., an expression level or TNF inhibitor signature score from an individual who is known to be a responder). In some cases, if the TNF inhibitor signature score is of the test individual is greater than the reference, the test individual can be predicted to be a non-responder to treatment with a TNF inhibitor. In some cases, if the TNF inhibitor signature score is of the test individual is less than or equal to the reference, the test individual can be predicted to be a responder to treatment with a TNF inhibitor.

On the other hand, an expression level or TNF inhibitor signature score of a test individual can be compared with a reference value (e.g., an expression level or TNF inhibitor signature score from an individual who is known to be a non-responder). In some cases, if the TNF inhibitor signature score is of the test individual is greater than or equal to the reference, the test individual can be predicted to be a non-responder to treatment with a TNF inhibitor. In some cases, if the TNF inhibitor signature score is of the test individual is less than the reference, the test individual can be predicted to be a responder to treatment with a TNF inhibitor.

In some cases, a prognosis can be made by comparing the expression level or TNF inhibitor signature score of the individual with a reference value that is a known threshold. For example, an expression level or TNF inhibitor signature score of an individual can be compared to reference values that are threshold values, where a score below (or in some cases equal to) the threshold is associated with a particular outcome (e.g., responder) and/or a score above (or in some cases equal to) the threshold is associated with a particular outcome (e.g., non-responder). In some cases, expression level or TNF inhibitor signature score may be compared to two different reference values (e.g., one known to be associated with responders and one known to be associated with non-responders) to obtain confirmed information regarding whether the individual is a responder or a non-responder.

In some cases, a prognosis is a statistical likelihood of predicted responsiveness to treatment with a TNF inhibitor. Such statistical likelihoods can be obtained by comparing an expression level or TNF inhibitor signature score from an individual to reference values from a set of individuals with varying levels of responsiveness to treatment with a TNF inhibitor. Such comparisons can be used to correlate a range of expression levels and/or TNF inhibitor signature scores a range of responsiveness likelihoods. Thus, expression level and/or TNF inhibitor signature score from an individual can be used to determine a statistical likelihood of responsiveness for the individual.

As another example, an expression level or TNF inhibitor signature score may be employed to monitor treatment with a TNF inhibitor. By “monitor treatment” with a TNF inhibitor, it is generally meant monitoring a subject's condition, e.g. to provide information as to the effect or efficacy of a TNF inhibitor treatment.

### Reports

In some embodiments, a report is generated. For example, in some cases a subject method includes a step of generating a report. A “report,” as described herein, is an electronic or tangible document which includes report elements that provide information of interest relating to the assessment of a subject and its results. In some embodiments, a subject report includes a measured expression level (e.g., a raw value, a normalized value, a normalized and weighted value, etc.) (e.g., an expression level of an RGS1 expression product and/or an expression level of an IL11 expression product) as discussed in greater detail above. In some embodiments, a subject report includes a calculated TNF inhibitor signature score for the individual from whom a biological sample was obtained (e.g., a TNF inhibitor signature score determined by calculating the geometric mean of an expression level of an RGS1 expression product and an expression level of an IL11 expression product). In some embodiments, a subject report includes an assessment (e.g. a prediction of whether the individual from whom the biological sample was obtained is a responder or non-responder to treatment with a TNF inhibitor, a treatment recommendation, a prescription, etc.).

A subject report can be completely or partially electronically generated. A subject report can also include one or more of: 1) information regarding a testing facility; 2) service provider information; 3) patient data; 4) sample data; 5) an assessment report, which can include various information including: a) reference values employed, and b) test data, where test data can include, e.g., an expression level determination for an RNA and/or a protein; and/or 6) other features.

In some embodiments, an assessment is provided by providing (e.g., generating) a report (e.g., a written report) that includes at least one of: (i) a measured expression level (e.g., a raw value, a normalized value, a normalized and weighted value, etc.) (e.g., an expression level of an RGS1 expression product and/or an expression level of an IL11 expression product); (ii) a TNF inhibitor signature score (e.g., determined by calculating the geometric mean of an expression level of an RGS1 expression product and an expression level of an IL11 expression product); (iii) a prediction of whether the individual from whom the biological sample was obtained is a responder or non-responder to treatment with a TNF inhibitor; (iv) a recommended treatment regimen (e.g., a recommendation to treat the individual with a TNF inhibitor, a recommendation not to treat the individual with a TNF inhibitor); and (v) a prescription for a treatment regimen. In some cases, the report can further include a reference value (or multiple reference values) (e.g., a reference value for an RGS1 expression product and/or an IL11 expression product; a reference value for a TNF inhibitor signature score, and the like).

Thus, the subject methods may include a step of generating or outputting a report, which report can be provided in the form of an electronic medium (e.g., an electronic display on a computer monitor), or in the form of a tangible medium (e.g., a report printed on paper or other tangible medium). Any form of report may be provided.

A report may include information about the testing facility, which information is relevant to the hospital, clinic, or laboratory in which sample gathering and/or data generation was conducted. Sample gathering can include obtaining a fluid sample, e.g. blood, saliva, urine etc.; a tissue sample, e.g. a tissue biopsy, etc. from a subject. Data generation can include (a) measuring an expression level of one or more expression products (e.g., an RGS1 expression product and/or an IL11 expression product) in patients, e.g., an individual that has an autoimmune and/or immune-mediated disorder (e.g., inflammatory bowel disease (IBD), psoriasis, plaque psoriasis, psoriatic arthritis, Crohn's disease (CD), ulcerative colitis (UC), rheumatoid arthritis (RA), ankylosing spondylitis, hidradenitis suppurativa, and refractory asthma), and/or (b) measuring an expression level of one or more expression products (e.g., an RGS1 expression product and/or an IL11 expression product) in one or more reference individuals, i.e. individuals that are already known to be responders or non-responders to treatment with TNF inhibitors. This information can include one or more details relating to, for example, the name and location of the testing facility, the identity of the lab technician who conducted the assay and/or who entered the input data, the date and time the assay was conducted and/or analyzed, the location where the sample and/or result data is stored, the lot number of the reagents (e.g., kit, etc.) used in the assay, and the like. Report fields with this information can generally be populated using information provided by the user.

The report may include information about the service provider, which may be located outside the healthcare facility at which the user is located, or within the healthcare facility. Examples of such information can include the name and location of the service provider, the name of the reviewer, and where necessary or desired the name of the individual who conducted sample gathering and/or data generation. Report fields with this information can generally be populated using data entered by the user, which can be selected from among pre-scripted selections (e.g., using a drop-down menu). Other service provider information in the report can include contact information for technical information about the result and/or about the interpretive report.

The report may include a patient data section, including patient medical history (which can include, e.g., age, race, serotype, current health, family medical history, and any other patient characteristics), as well as administrative patient data such as information to identify the patient (e.g., name, patient date of birth (DOB), gender, mailing and/or residence address, medical record number (MRN), room and/or bed number in a healthcare facility), insurance information, and the like), the name of the patient's physician or other health professional who ordered the monitoring assessment and, if different from the ordering physician, the name of a staff physician who is responsible for the patient's care (e.g., primary care physician).

The report can include a sample data section, which may provide information about the biological sample analyzed in the monitoring assessment, such as the source of biological sample obtained from the patient (e.g. Tumor, blood, saliva, or type of tissue, etc.), how the sample was handled (e.g. storage temperature, preparatory protocols) and the date and time collected. Report fields with this information can generally be populated using data entered by the user, some of which may be provided as pre-scripted selections (e.g., using a drop-down menu). The report may include a results section. For example, the report may include a section reporting the results of measuring expression level(s) and/or a calculated TNF inhibitor signature score.

The report may include an assessment report section, which may include information generated after processing of the data as described herein. The interpretive report can include a prognosis of TNF inhibitor treatment (e.g., a prediction of whether the individual is a responder or non-responder to treatment with a TNF inhibitor). The assessment portion of the report can optionally also include a recommendation(s) (e.g., recommendation as to whether the individual should be treated with a TNF inhibitor). For example, where the results indicate that the individual is a responder, the recommendation can include a recommendation for therapy to include administration of a TNF inhibitor; and where the results indicate that the individual is a non-responder, the recommendation can include a recommendation for therapy not to include administration of a TNF inhibitor.

It will also be readily appreciated that the reports can include additional elements or modified elements. For example, where electronic, the report can contain hyperlinks which point to internal or external databases which provide more detailed information about selected elements of the report. For example, the patient data element of the report can include a hyperlink to an electronic patient record, or a site for accessing such a patient record, which patient record is maintained in a confidential database. This latter embodiment may be of interest in an in-hospital system or in-clinic setting. When in electronic format, the report is recorded on a suitable physical medium, such as a computer readable medium, e.g., in a computer memory, zip drive, CD, DVD, etc.

It will be readily appreciated that the report can include all or some of the elements above, with the proviso that the report generally includes at least one of: (i) one or more measured expression levels (e.g., one or more raw values, normalized values, normalized and weighted values, etc.) (e.g., an expression level of an RGS1 expression product and/or an expression level of an IL11 expression product); (ii) a TNF inhibitor signature score (e.g., determined by calculating the geometric mean of an expression level of an RGS1 expression product and an expression level of an IL11 expression product); (iii) a prediction of whether the individual from whom the biological sample was obtained is a responder or non-responder to treatment with a TNF inhibitor; (iv) a recommended treatment regimen (e.g., a recommendation to treat the individual with a TNF inhibitor, a recommendation not to treat the individual with a TNF inhibitor); and (v) a prescription for a treatment regimen. In some cases, the report can further include a reference value (or multiple reference values) (e.g., a reference value for an RGS1 expression product and/or an IL11 expression product; a reference value for a TNF inhibitor signature score, and the like). As noted above, in some cases, the report can further include a reference value (or multiple reference values) (e.g., a reference value for an RGS1 expression product and/or an IL11 expression product; a reference value for a TNF inhibitor signature score, and the like).

### Biological Samples

Aspects of the disclosure include measuring expression levels in a biological sample from an individual. The terms “recipient”, “individual”, “subject”, “host”, and “patient”, are used interchangeably herein and refer to any mammalian subject for whom measurement of expression levels, prognosis, diagnosis, prediction, treatment, and/or therapy is desired. “Mammal” for purposes of treatment refers to any animal classified as a mammal, including humans, domestic and farm animals, and zoo, sports, or pet animals, such as dogs, horses, cats, cows, sheep, goats, pigs, camels, etc. In some embodiments, the individual of a subject method is human.

The term “biological sample” encompasses a variety of sample types obtained from an organism and can be used in a diagnostic, prognostic, or monitoring assay. The term encompasses blood and other liquid samples of biological origin or cells derived therefrom and the progeny thereof. The term encompasses samples that have been manipulated in any way after their procurement, such as by treatment with reagents, solubilization, or enrichment for certain components. The term encompasses a clinical sample, and also includes cell supernatants, cell lysates, serum, plasma, biological fluids, and tissue samples (e.g., tissue taken from a site of inflammation, a biopsy, and the like). Clinical samples for use in the methods of the invention may be obtained from a variety of sources including, but not limited to tissue from a site of inflammation, a biopsy sample, a thoracentesis sample, a fine needle aspirate, and the like. Exemplary biological samples include, but are not limited to: a suspension of cells (e.g., from a peripheral blood sample, an aspirate, a cell suspension from tissue isolated from a site of inflammation, a cell suspension from a biopsy sample, etc.), a biopsy, an aspirate (e.g., a fine needle aspirate, a thoracentesis sample, etc.), a fixed tissue sample (e.g., a formalin-fixed paraffin embedded (FFPE) tissue sample, an FFPE biopsy sample, etc.), and a homogenized tissue (e.g., a homogenized tissue sample where the tissue is from a site of inflammation, a homogenized biopsy sample, a homogenized paraffin- or OCT-embedded sample, etc.).

Once a sample is isolated (i.e., collected), it can be used directly, frozen, or maintained in appropriate culture medium for a period of time (e.g., in some cases, an extended period of time). Typically the samples will be from human patients, although animal models may find use, e.g. equine, bovine, porcine, canine, feline, rodent, e.g. mice, rats, hamster, primate, etc. Any convenient tissue sample that demonstrates differential representation of the one or more markers disclosed herein (an RGS1 expression product and/or an IL11 expression product) among individuals who are non-responsive versus responsive to treatment with a TNF inhibitor can be evaluated in the subject methods.

The subject sample can be treated in a variety of ways so as to enhance detection of the expression products. For example, where the sample is taken from a site of inflammation, non-immune cells (or particular types of immune cells) may be removed from the sample (e.g., by differential centrifugation, by differential binding and/or labeling, e.g., FACs sorting and/or magnetic separation techniques) prior to assaying. For example, where the sample is a tumor sample (e.g., a biopsy), non-tumor cells may be removed from the sample (e.g., by differential centrifugation, by differential binding and/or labeling, e.g., FACs sorting and/or magnetic separation techniques) prior to assaying. Where the sample is blood, the red blood cells may be removed from the sample (e.g., by centrifugation) prior to assaying. Such a treatment may serve to reduce the non-specific background levels of detecting an expression level of an expression product. Measurement of an expression level may also be enhanced by concentrating the sample using procedures well known in the art (e.g. acid precipitation, alcohol precipitation, salt precipitation, hydrophobic precipitation, filtration (using a filter which is capable of retaining molecules greater than 30 kD, e.g. Centrim 30™), affinity purification, etc.). In some embodiments, the pH of the test and control samples can be adjusted to, and maintained at, a pH which approximates neutrality (i.e. pH 6.5-8.0). Such a pH adjustment can prevent complex formation, thereby providing a more accurate quantitation of the level of expression product in the sample. In embodiments where the sample is urine, the pH of the sample can be adjusted and the sample can be concentrated in order to enhance the detection of the marker.

### Reagents, Systems and Kits

Also provided are reagents, systems and kits thereof for practicing one or more of the above-described methods. The subject reagents, systems and kits thereof may vary greatly. Reagents of interest include reagents specifically designed for use in measuring expression levels and/or calculating a TNF inhibitor signature score, for example, one or more detection elements (e.g. oligonucleotides for the detection of nucleic acids, e.g., primers, probes, etc.; antibodies or peptides for the detection of protein; and the like). In some instances, the detection element comprises a reagent to detect the expression of RGS1. In some instances, the detection element comprises a reagent to detect the expression of IL11. In some instances, the detection element comprises a reagent to detect the expression of RGS1 and IL11. For example, the detection element may be a dipstick, a plate, an array, or cocktail that comprises one or more detection elements, e.g. one or more oligonucleotides, one or more sets of PCR primers, one or more antibodies, etc. which may be used to measure the expression level of RGS1 and/or IL11 expression products.

Is some cases, a reagent is a collection of antibodies that bind specifically to RGS1 and IL11, e.g. in an ELISA format, in an xMAP™ microsphere format, on a proteomic array, in suspension for analysis by flow cytometry, by western blotting, by dot blotting, or by immunohistochemistry. Methods for using the same are well understood in the art. These antibodies can be provided in solution. Alternatively, they may be provided pre-bound to a solid matrix, for example, the wells of a multi-well dish or the surfaces of xMAP microspheres.

Is some cases, a reagent is an array of probe nucleic acids in which the genes of interest are represented. A variety of different array formats are known in the art, with a wide variety of different probe structures, substrate compositions and attachment technologies (e.g., dot blot arrays, microarrays, etc.). Representative array structures of interest include those described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280.

Is some cases, a reagent is a collection of gene specific primers that designed to selectively amplify RGS1 and/or IL11 expression products (e.g., using a PCR-based technique, e.g., real-time RT-PCR). Of interest are arrays of probes, collections of primers, or collections of antibodies that include probes, primers or antibodies (also called reagents) that are specific for RGS1. Of interest are arrays of probes, collections of primers, or collections of antibodies that include probes, primers or antibodies (also called reagents) that are specific for IL11. Of interest are arrays of probes, collections of primers, or collections of antibodies that include probes, primers or antibodies (also called reagents) that are specific for RGS1 and IL11.

Procedures using these kits can be performed by clinical laboratories, experimental laboratories, medical practitioners, or private individuals. The kits of the invention may comprise amplification and/or sequencing primers, and/or hybridization primers or antibodies for protein determination. The kit may optionally provide additional components that are useful in the procedure, including, but not limited to, buffers, developing reagents, labels, reacting surfaces, means for detection, control samples, standards, instructions, and interpretive information.

Kits of the subject disclosure may include the above-described arrays, gene-specific primers (e.g., primer collections), or protein-specific antibody collections. Kits may further include one or more additional reagents employed in the various methods, such as primers for generating target nucleic acids, dNTPs and/or rNTPs, which may be either premixed or separate, one or more uniquely labeled dNTPs and/or rNTPs, such as biotinylated or Cy3 or Cy5 tagged dNTPs, gold or silver particles with different scattering spectra, or other post synthesis labeling reagent, such as chemically active derivatives of fluorescent dyes, enzymes, such as reverse transcriptases, DNA polymerases, RNA polymerases, and the like, various buffer mediums, e.g. hybridization and washing buffers, prefabricated probe arrays, labeled probe purification reagents and components, like spin columns, etc., signal generation and detection reagents, e.g. labeled secondary antibodies, streptavidin-alkaline phosphatase conjugate, chemifluorescent or chemiluminescent substrate, and the like.

The subject kits may also include one or more prediction elements, which element is can be a reference or control sample or reference value that can be employed, e.g., by a suitable experimental or computing means, to make a prediction of responsiveness to TNF inhibitor treatment based on an “input” marker level profile (e.g., an ‘input’ of a measured expression level and/or calculated TNF inhibitor signature score from a sample from an individual). Representative prediction elements include samples from an individual known to be responsive or non-responsive to TNF inhibitor treatment; reference values for expression levels of RGS1 and/or IL11 and/or a TNF inhibitor signature score that are associated with responsiveness or non-responsiveness to TNF inhibitor treatment; and the like.

In addition to the above components, the subject kits can further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, hard-drive, network data storage, etc., on which the information has been recorded. Yet another means that may be present is a website address which may be used via the internet to access the information at a removed site. Any convenient means may be present in the kits.

In addition to instructions for using the components of the kit, the kit can further include instructions of analyzing the data acquired from the assays described herein. For example, the instructions can include a graph and/or table of known statistics for the probabilities of being responsive or non-responsive to TNF inhibitor treatment for individuals having differing expression levels of RGS1 and/or IL11. In addition, instructions can be provided to interpret these graphs and/or tables. These graphs and/or tables and instructions would be generally recorded on a suitable recording medium, for example, printed on a substrate such as paper or plastic. Alternatively, these graphs and/or tables and instructions can be provided on an electronic storage data file present on a suitable computer readable storage medium, e.g. CD-ROM, diskette, etc. In some embodiments, the actual graphs and/or table and instructions are not present in the kit, but means for obtaining the graphs/tables and instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### Computer-Implemented Methods, Systems and Devices

The methods of the present disclosure can be computer-implemented, such that method steps (e.g., assaying (e.g., measuring), calculating, comparing, predicting, reporting, and the like) can be automated in whole or in part. Accordingly, the present disclosure provides methods, computer systems, devices and the like in connection with computer-implemented methods of predicting whether an individual will respond to treatment with a TNF inhibitor, and/or methods of determining a treatment regimen for an individual.

For example, the method steps, including measuring an expression level of an RGS1 expression product and/or an expression level of an IL11 expression product in a biological sample from an individual, calculating a TNF inhibitor signature score for the individual (e.g., calculating a geometric mean of said measured expression levels to obtain a TNF inhibitor signature score for the individual), comparing a measured expression level and/or calculated score to a reference value (e.g., determining that a measured expression level or a TNF inhibitor signature score is less than a reference value, determining that a measured expression level or a TNF inhibitor signature score is ‘less than or equal to’ a reference value, determining that a measured expression level or a TNF inhibitor signature score is greater than a reference value, determining that a measured expression level or a TNF inhibitor signature score is ‘greater than or equal to’ a reference value, and the like), generating a report, and the like, can be completely or partially performed by a computer program product. Values obtained can be stored electronically, e.g., in a database, and can be subjected to an algorithm executed by a programmed computer.

For example, the methods of the present disclosure can involve inputting the expression levels (e.g. raw values, normalized values, weighted values, and/or normalized and weighted values) of an RGS1 expression product and/or an IL11 expression product into a computer programmed to execute an algorithm to perform the comparing step (e.g., determining that a measured expression level or a TNF inhibitor signature score is less than a reference value, determining that a measured expression level or a TNF inhibitor signature score is ‘less than or equal to’ a reference value, determining that a measured expression level or a TNF inhibitor signature score is greater than a reference value, determining that a measured expression level or a TNF inhibitor signature score is ‘greater than or equal to’ a reference value, and the like), and generate a report as described herein, e.g., by displaying or printing a report to an output device at a location local or remote to the computer.

The present invention thus provides a computer program product including a computer readable storage medium (e.g., a nontransitory computer-readable storage medium) having a computer program stored on it. The program can, when read by a computer, execute relevant calculations based on values obtained from analysis of one or more biological samples from an individual. The computer program product has stored therein a computer program for performing the calculation(s).

The present disclosure provides systems for executing the program described above, which system generally includes: (i) a central computing environment; (ii) an input device, operatively connected to the computing environment, to receive patient data (e.g., expression level data, clinical data from the patient/individual, etc. as described above); (iii) an output device, connected to the computing environment, to provide information to a user (e.g., medical personnel, clinician, and the like); and (iv) an algorithm executed by the central computing environment (e.g., a processor), where the algorithm is executed based on the data received by the input device, and where the algorithm can in some cases calculate a value and/or category, which value and/or category is indicative of (can be used to predict) whether an individual is responsive or non-responsive to treatment with a TNF inhibitor.

### Systems

In some cases, a subject system includes (I) a first system (e.g., a biomolecule analyzing system) that performs a measuring/detection step to generate a value which represents an expression level of a subject expression product, and (II) a second system that is a computer system. The first and second systems are integrated into a system by virtue of the first system passing the measured expression level data to the second system for analysis. Any convenient measuring/detection system can be used and many suitable systems will be known to one of ordinary skill in the art. While some biomolecule analyzing systems can be considered to be a nucleic acid analyzing system (e.g., a thermocyler, a nucleic acid sequencing machine, and the like), and other biomolecule analyzing systems can be considered to be a protein analyzing system (e.g., an automated ELISA analyzer such as a plate reader, a mass spectrometer, and the like), yet other biomolecule analyzing systems can be used as both a nucleic acid and protein analyzing system (e.g., a flow cytometer). Thus, the term “biomolecule analyzing system” encompasses systems that analyze nucleic acids (e.g., measure levels of nucleic acids in a sample) and systems that analyze proteins (e.g., measure levels of proteins in a sample), as well as systems that analyze both nucleic acids and proteins (e.g., measure levels of nucleic acids and/or proteins in a sample).

A biomolecule analyzing system (e.g., a nucleic acid analyzing system, a protein analyzing system) includes (a) a detector for measuring/detecting a target biomolecule (e.g., an RNA, a protein)(e.g., for measuring an expression level of an RGS1 expression product and/or an expression level of an IL11 expression product), where the detector is coupled to a computer system (e.g., a computer system that can process the data measured by the detector). Thus, the biomolecule analyzing system can measure an expression level of an RGS1 expression product and/or an expression level of an IL11 expression product, and can then send the measured expression levels to the computer system (the second system).

A biomolecule analyzing system can included a wide variety of different detectors, depending on the labels and assays. Examples of useful detectors include but are not limited to: a microscope(s) (e.g., with multiple channels of fluorescence); a plate reader (e.g., to provide fluorescent, ultraviolet, and/or visible spectrophotometric detection); a CCD camera that can capture data images and transform them into quantifiable formats; etc.

A biomolecule analyzing system can further include liquid handling components (e.g., a robotic systems that includes any number of components). Liquid handling components can be partially or fully automated. A wide variety of components which can be used, including, but not limited to, one or more robotic arms; plate handlers for the positioning of microplates; automated lid or cap handlers to remove and replace lids for wells; tip assemblies for sample distribution with disposable tips; washable tip assemblies for sample distribution; 96 well loading blocks; cooled reagent racks; microtitler plate pipette positions (optionally cooled); stacking towers for plates and tips; etc. Fully robotic or microfluidic systems can include automated liquid-, particle-, cell- and organism-handling including high throughput pipetting to perform all steps of screening applications. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration.

Examples of biomolecule analyzing systems include but are not limited to: a flow cytometer (which can function as a nucleic acid analyzing system and/or a protein analyzing system), a thermocycler (e.g., a nucleic acid analyzing system for assays such as qRT-PCR), a mass spectrophotometer (a protein analyzing system), and a Next Generation high-throughput sequencer (a nucleic acid analyzing system).

### Computer Systems

The present disclosure provides computer systems for calculating a TNF inhibitor signature score for an individual, and/or for providing a prediction of for an individual (e.g., a prediction as to whether the individual is/will be responsive or non-responsive to treatment with a TNF inhibitor). The computer systems include a processor and memory operably coupled to the processor, where the memory programs the processor to perform at least one of the following tasks: receive assay data (e.g., expression level of an RGS1 expression product and/or an IL11 expression product) from a biological sample from an individual; calculate a TNF inhibitor signature score; compare the expression level(s) and or the TNF inhibitor signature score with a reference (e.g., determine that a measured expression level or a TNF inhibitor signature score is less than a reference value, determine that a measured expression level or a TNF inhibitor signature score is ‘less than or equal to’ a reference value, determine that a measured expression level or a TNF inhibitor signature score is greater than a reference value, determine that a measured expression level or a TNF inhibitor signature score is ‘greater than or equal to’ a reference value, and the like); and provide a prediction for the individual (e.g., a prediction as to whether the individual is/will be responsive or non-responsive to treatment with a TNF inhibitor).

Computer systems may include a processing system, which generally comprises at least one processor or processing unit or plurality of processors, memory, at least one input device and at least one output device, coupled together via a bus or group of buses. In certain embodiments, an input device and output device can be the same device. The memory can be any form of memory device, for example, volatile or non-volatile memory, solid state storage devices, magnetic devices, etc. The processor can comprise more than one distinct processing device, for example to handle different functions within the processing system.

An input device receives input data and can comprise, for example, a keyboard, a pointer device such as a pen-like device or a mouse, audio receiving device for voice controlled activation such as a microphone, data receiver or antenna such as a modem or wireless data adaptor, data acquisition card, etc. Input data can come from different sources, for example keyboard instructions in conjunction with data received via a network.

Output devices produce or generate output data and can comprise, for example, a display device or monitor in which case output data is visual, a printer in which case output data is printed, a port for example a USB port, a peripheral component adaptor, a data transmitter or antenna such as a modem or wireless network adaptor, etc. Output data can be distinct and derived from different output devices, for example a visual display on a monitor in conjunction with data transmitted to a network. A user can view data output, or an interpretation of the data output, on, for example, a monitor or using a printer. The storage device can be any form of data or information storage means, for example, volatile or non-volatile memory, solid state storage devices, magnetic devices, etc.

In use, the processing system may be adapted to allow data or information to be stored in and/or retrieved from, via wired or wireless communication means, at least one database. The interface may allow wired and/or wireless communication between the processing unit and peripheral components that may serve a specialized purpose. In general, the processor can receive instructions as input data via input device and can display processed results or other output to a user by utilizing output device. More than one input device and/or output device can be provided. A processing system may be any suitable form of terminal, server, specialized hardware, or the like.

A processing system may be a part of a networked communications system. A processing system can connect to a network, for example the Internet or a WAN. Input data and output data can be communicated to other devices via the network. The transfer of information and/or data over the network can be achieved using wired communications means or wireless communications means. A server can facilitate the transfer of data between the network and one or more databases. A server and one or more databases provide an example of an information source.

Thus, a processing computing system environment may operate in a networked environment using logical connections to one or more remote computers. The remote computer may be a personal computer, a server, a router, a network PC, a peer device, or other common network node, and typically includes many or all of the elements described above.

Certain embodiments may be described with reference to acts and symbolic representations of operations that are performed by one or more computing devices. As such, it will be understood that such acts and operations, which are at times referred to as being computer-executed, include the manipulation by the processor of the computer of electrical signals representing data in a structured form. This manipulation transforms the data or maintains them at locations in the memory system of the computer, which reconfigures or otherwise alters the operation of the computer in a manner understood by those skilled in the art. The data structures in which data is maintained are physical locations of the memory that have particular properties defined by the format of the data. However, while an embodiment is being described in the foregoing context, it is not meant to be limiting as those of skill in the art will appreciate that the acts and operations described hereinafter may also be implemented in hardware.

Embodiments may be implemented with numerous other general-purpose or special-purpose computing devices and computing system environments or configurations. Examples of well-known computing systems, environments, and configurations that may be suitable for use with an embodiment include, but are not limited to, personal computers, handheld or laptop devices, personal digital assistants, multiprocessor systems, microprocessor-based systems, programmable consumer electronics, network, minicomputers, server computers, web server computers, mainframe computers, and distributed computing environments that include any of the above systems or devices.

Embodiments may be described in a general context of computer-executable instructions, such as program modules, being executed by a computer. Generally, program modules include routines, programs, objects, components, data structures, etc., that perform particular tasks or implement particular abstract data types. An embodiment may also be practiced in a distributed computing environment where tasks are performed by remote processing devices that are linked through a communications network. In a distributed computing environment, program modules may be located in both local and remote computer storage media including memory storage devices.

### Computer Program Products

The present disclosure provides computer program products that, when executed on a programmable computer such as that described above, can carry out the methods of the present disclosure. As discussed above, the subject matter described herein may be embodied in systems, apparatus, methods, and/or articles depending on the desired configuration. These various implementations may include implementation in one or more computer programs that are executable and/or interpretable on a programmable system including at least one programmable processor, which may be special or general purpose, coupled to receive data and instructions from, and to transmit data and instructions to, a storage system, at least one input device (e.g. video camera, microphone, joystick, keyboard, and/or mouse), and at least one output device (e.g. display monitor, printer, etc.).

Computer programs (also known as programs, software, software applications, applications, components, or code) include instructions for a programmable processor, and may be implemented in a high-level procedural and/or object-oriented programming language, and/or in assembly/machine language. As used herein, the term “machine-readable medium” refers to any nontransitory computer program product, apparatus and/or device (e.g., magnetic discs, optical disks, memory, etc.) used to provide machine instructions and/or data to a programmable processor, including a machine-readable medium that receives machine instructions as a machine-readable signal.

It will be apparent from this description that aspects of the present invention may be embodied, at least in part, in software, hardware, firmware, or any combination thereof. Thus, the techniques described herein are not limited to any specific combination of hardware circuitry and/or software, or to any particular source for the instructions executed by a computer or other data processing system. Rather, these techniques may be carried out in a computer system or other data processing system in response to one or more processors, such as a microprocessor, executing sequences of instructions stored in memory or other computer-readable medium including any type of ROM, RAM, cache memory, network memory, floppy disks, hard drive disk (HDD), solid-state devices (SSD), optical disk, CD-ROM, and magnetic-optical disk, EPROMs, EEPROMs, flash memory, or any other type of media suitable for storing instructions in electronic format.

In addition, the processor(s) may be, or may include, one or more programmable general-purpose or special-purpose microprocessors, digital signal processors (DSPs), programmable controllers, application specific integrated circuits (ASICs), programmable logic devices (PLDs), trusted platform modules (TPMs), or the like, or a combination of such devices. In alternative embodiments, special-purpose hardware such as logic circuits or other hardwired circuitry may be used in combination with software instructions to implement the techniques described herein.

The invention now being fully described, it will be apparent to one of ordinary skill in the art that various changes and modifications can be made without departing from the spirit or scope of the invention.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

The following example demonstrates the development of a method based on meta-analysis of gene expression data to identify biomarkers that translate across multiple diseases. As a proof of concept, an initial disease-specific signature of robust biomarkers in IBD was identified by training and validating on independent datasets. The disease-specific component of the signature was subsequently removed by integrating a gene expression model of IBD, thus creating a disease-independent signature. The disease-independent signature can include the expression products (e.g., RNA, protein) of two genes (e.g., IL11 and/or RGS1).

The disease-independent signature performs equally well as the original IBD-specific signature when predicting which IBD patients will respond to Infliximab (a TNFi). Finally, the performance of the disease-centered and disease-independent signatures were compared in a different type of autoimmune disorder (Psoriasis), and a marked and statistically significant improvement (AUC=0.77 whole signature; AUC=0.91 disease-independent signature, p-value=0.00140) was observed. Thus, the methods provided herein further demonstrate that biomarkers can be applied to multiple diseases (e.g., the disease-independent signature disclosed here can be applied to multiple diseases).

### Results

### Construction and Testing of a Disease-Centered Drug Response Signature

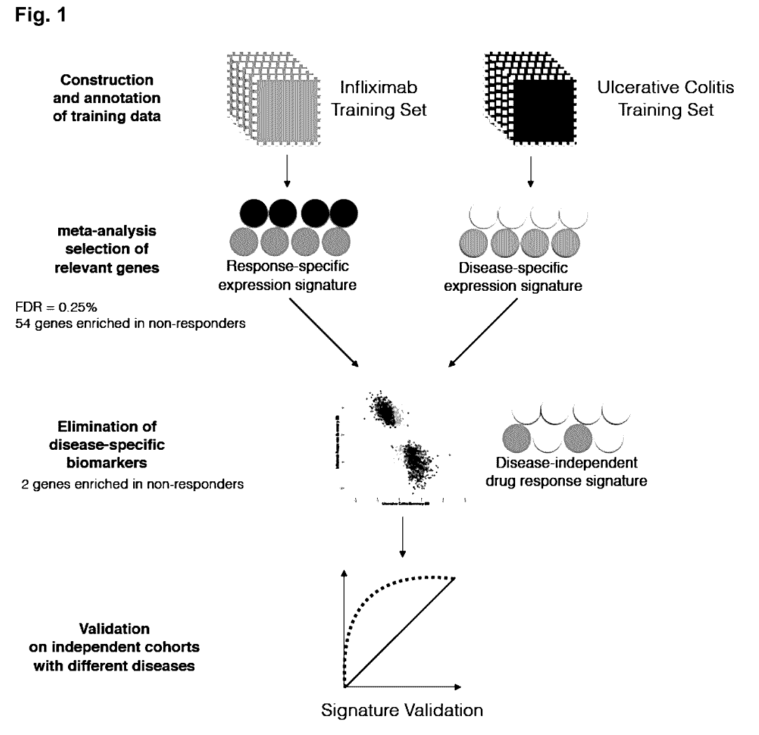

A TNF-response gene signature was built using publicly available gene expression datasets. Briefly, microarray gene expression data from colon biopsy samples collected at baseline was annotated for two cohorts of patients afflicted by Ulcerative Colitis, a clinical type of IBD (FIG. 1; GSE12251 and GSE14850). After biopsy, the patients had received Infliximab, a common TNF inhibitor, and clinical response was assessed 14 weeks after therapy. These cohorts were used as a discovery set to build a signature.

By applying meta-analysis to gene expression data (e.g., see Khatri P et al J. Exp. Medicine 2013, Sweenie T et al in print 2015), a multi-cohort gene expression signature for Infliximab response in Ulcerative Colitis was derived (see methods for details). For each gene, we associated a summary effect size, a statistical score derived by integrating information from all the training datasets, and a measure of statistical significance by FDR. The signature was filtered in order to select a final number of significant genes between 50 to 100. This number of genes was chosen in order to have a selective yet large enough set to ensure robustness in further statistical analysis. A “disease-centered” signature of 54 genes (FDR=0.25%) was obtained.

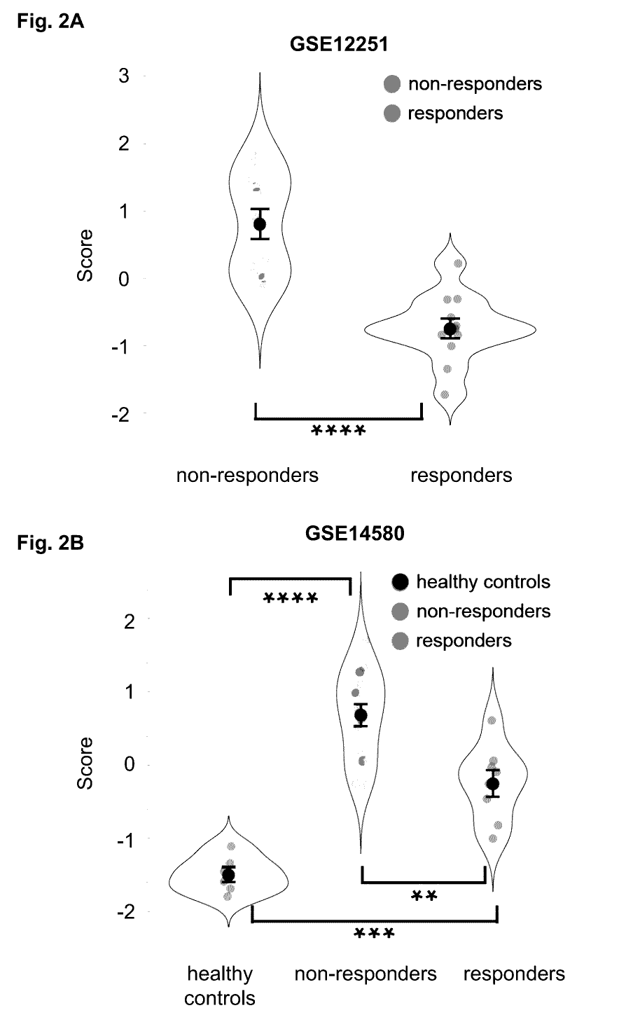

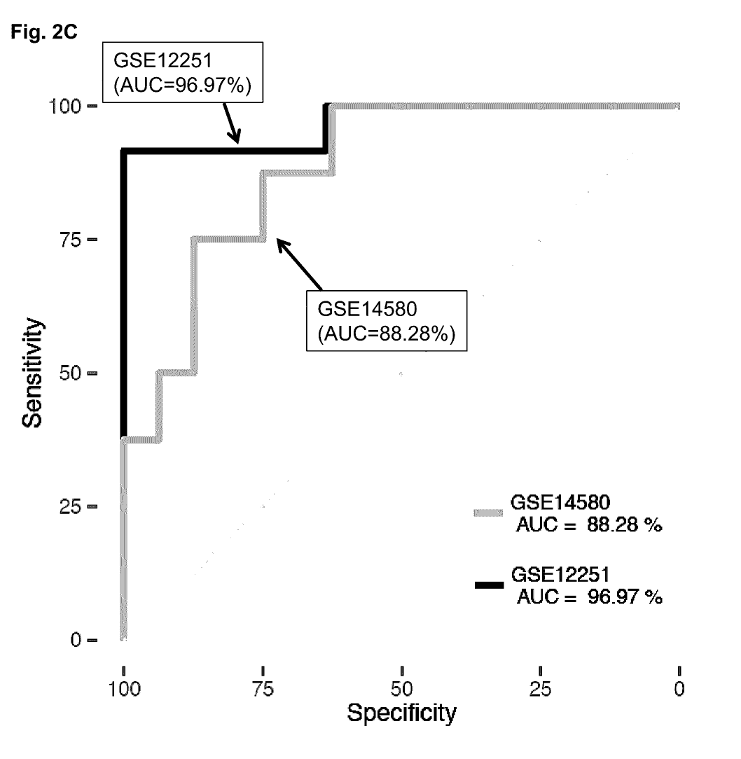

It was then tested whether the list of biomarkers could classify responders from non-responders. For this purpose, a signature score was computed for each patient, where the signature score consisted of the geometric mean of the expression value of the selected genes. The disease-centered signature robustly distinguished responders from non-responders in the training set (AUC=88.28% GSE14850; AUC=96.97% GSE12251; for details see methods, FIG. 2A-2C).

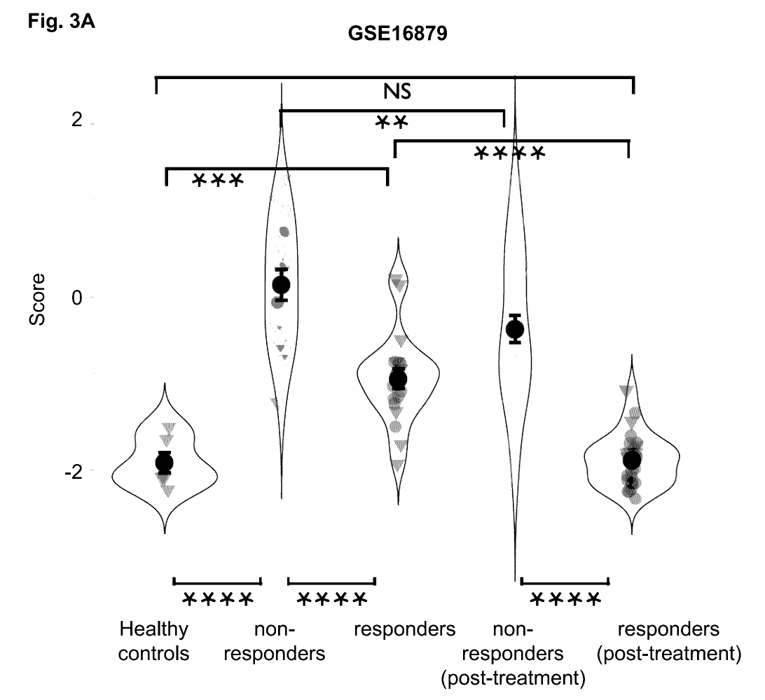

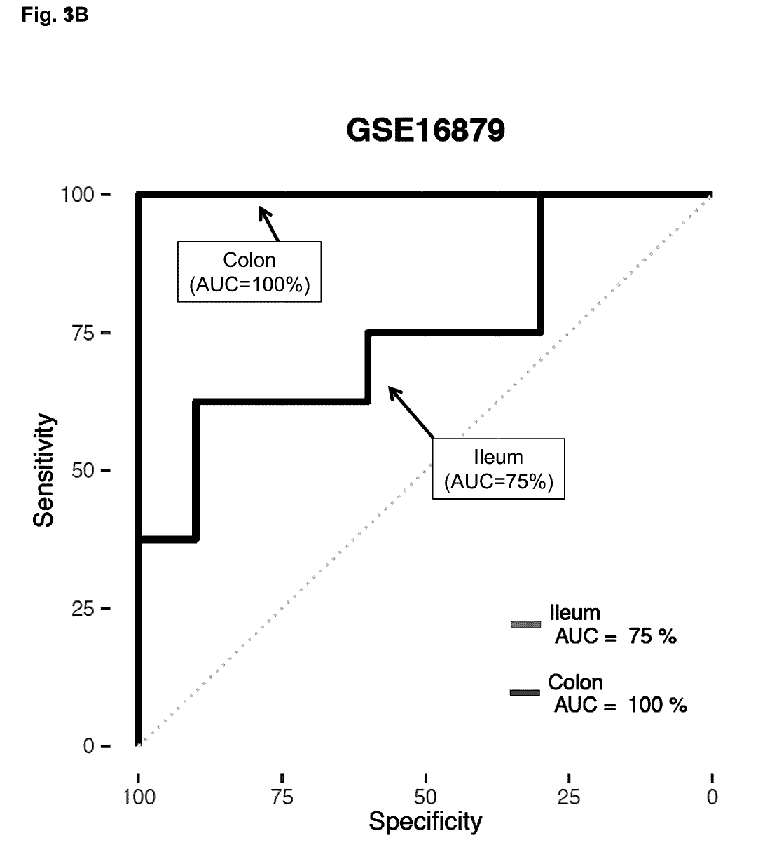

The signature was then tested on an independent validation cohort of colon and ileum biopsies of patients affected by Crohn's Disease, a different clinical type of IBD (GSE16879). The disease-centered signature perfectly classified responders from non-responders at baseline in colon (AUC=100%) and showed good performance in ileum (AUC=75%) (FIG. 3A-3B). This result suggests that the signature is robust and did not overfit the training cohorts. These results indicate that the signature is robust and consistent across different datasets within the same disease type.

Selection of Disease-Independent Biomarkers from Drug-Response Signature

In order to be able to predict drug-response in multiple conditions, the goal was to generalized the disease-centered signature. To do so, it was assessed whether the diseased-centered signature depended on the underlying disease state of the patient. No significant difference in Mayo Clinic Score between responders and non-responders had been observed in the training cohorts, suggesting that symptoms and morphology are not associated with the signature.

To see whether this was reflected at a molecular level, a gene expression signature for Ulcerative Colitis was generated to serve as a background model for disease activity and then compared it to the Infliximab response signature. Publicly available gene expression datasets were annotated, where the gene expression datasets measured gene expression from colon biopsies of patients affected by Ulcerative Colitis compared to healthy controls (see methods for details). Meta-analysis was applied to generate a gene expression signature of disease.

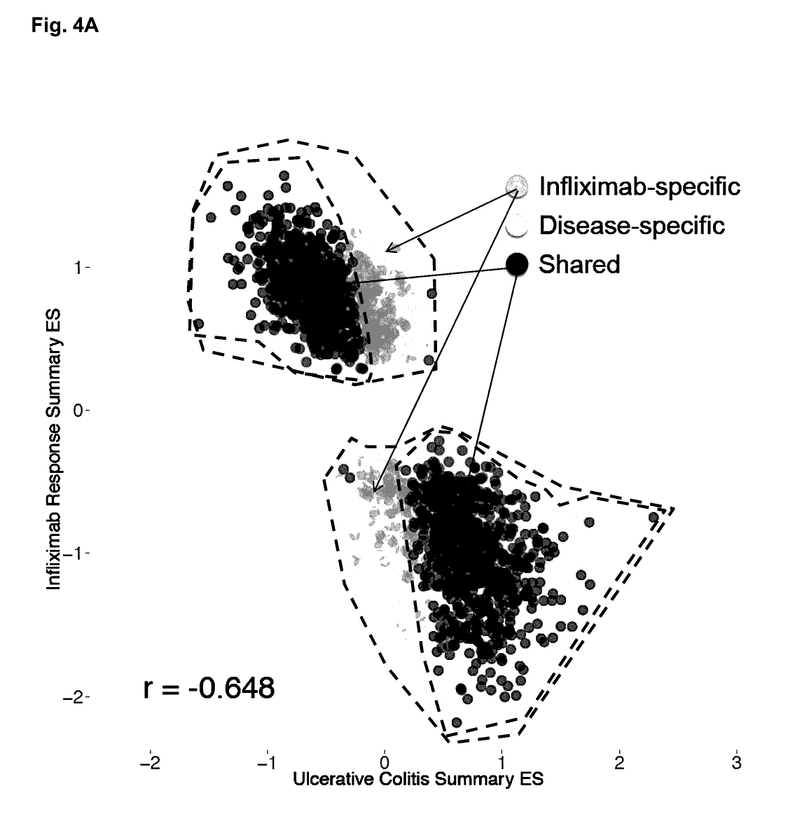

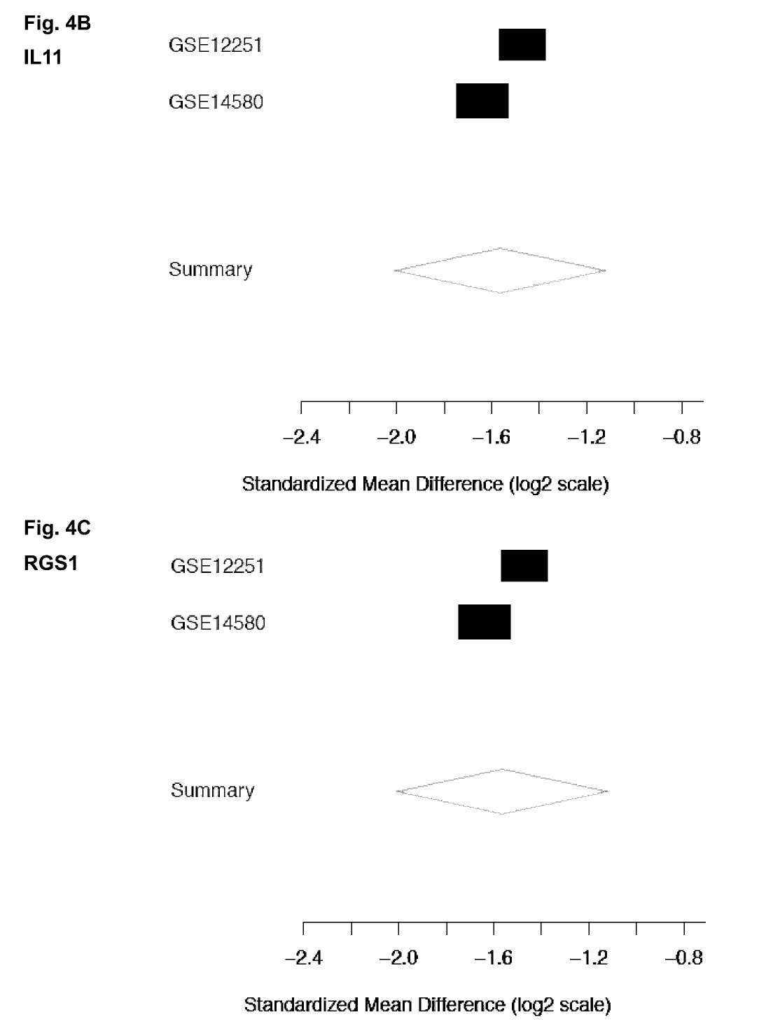

In contrast to the clinical scores, a strong negative correlation between the gene expression signatures for Infliximab response and Ulecrative Colitis (r=−0.648) was observed. This indicates that the disease-centered signature is in fact strongly associated with the molecular state of the disease, where a stronger disease score would indicate an impaired ability to respond to treatment (FIG. 4A).

This observation was validated by analyzing samples from the IBD validation set that were collected from control patients without the disease as well as IBD patients after Infliximab treatment (FIG. 3A). After treatment, responders did not show any significant difference in signature score when compared to control patients, but non-responders did (p>0.05 responders; p<10⁻⁴ non-responders). Furthermore, treatment produced a significant increase in score after therapy for responders and non responders respectively (p<0.01 non-responders; p<0.001 responders, paired-t-test).

This result supports the finding that treatment induces a molecular state closer to a non-diseased patient, and that non-responders show a higher level of disease score compared to responders. By performing FDR (false discovery rate) thresholding on both signatures, 3 cluster of genes were identified: a group of genes significantly associated with disease but not drug response (FIG. 4A: yellow set), a group of genes significantly associated with drug-response but not disease (FIG. 4A: cyan set), and a group of genes significant in both disease and drug response (FIG. 4A: green set).

A “disease-independent” signature was created by intersecting the disease-centered signature with a set of all genes that were not significantly associated with IBD (cyan set FIG. 4A). The disease-independent signature consists of two genes, IL11 and RGS1, both strongly enriched in non-responders (FIG. 4B-4C).

### Performance of Disease-Independent Biomarkers in Validation Sets

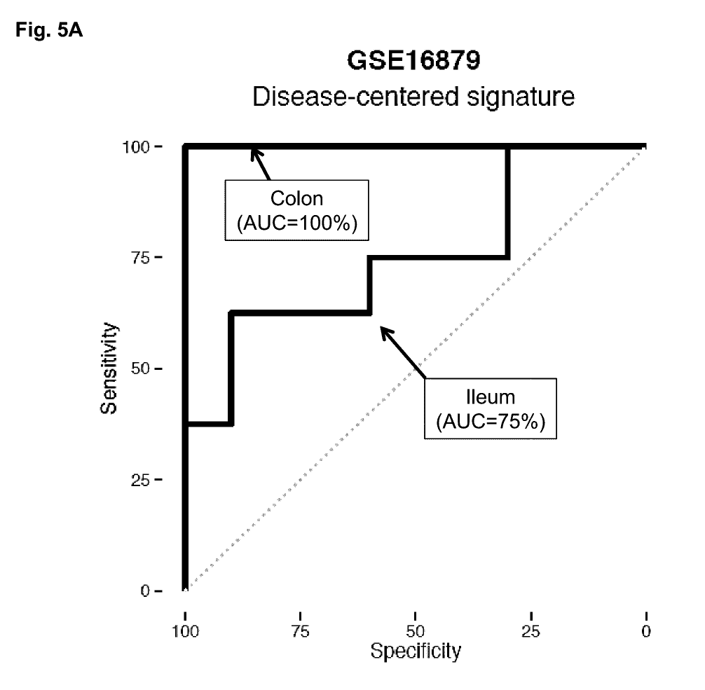

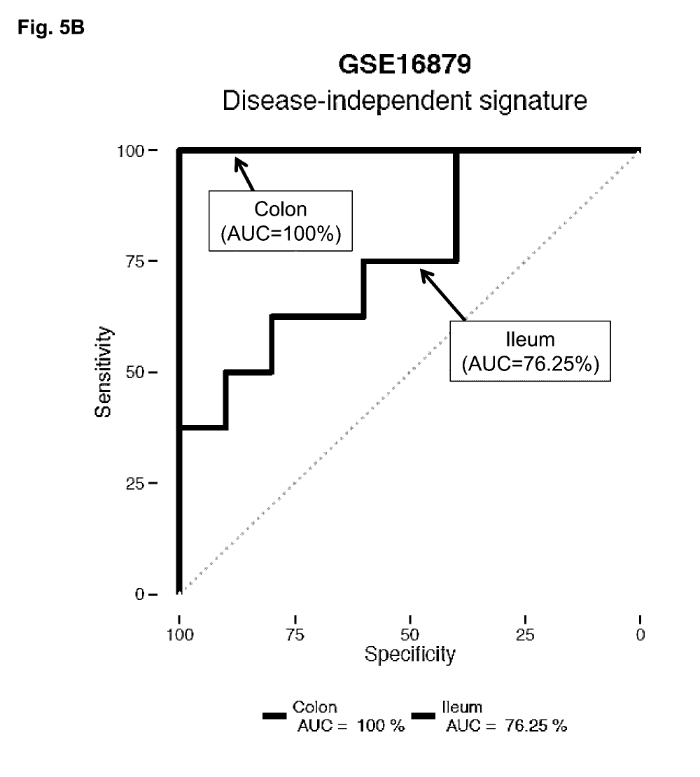

The performance of the disease-independent-signature was assessed on the IBD validation dataset (GSE16879) (FIG. 5A-5B). The disease-independent signature performed identically to the disease-centered signature in colon (AUC=100%, FIG. 5B) and showed a minor increase in performance in ileum (AUC=76.25%, FIG. 5B) despite the fact that the number of genes was reduced from 54 to 2.

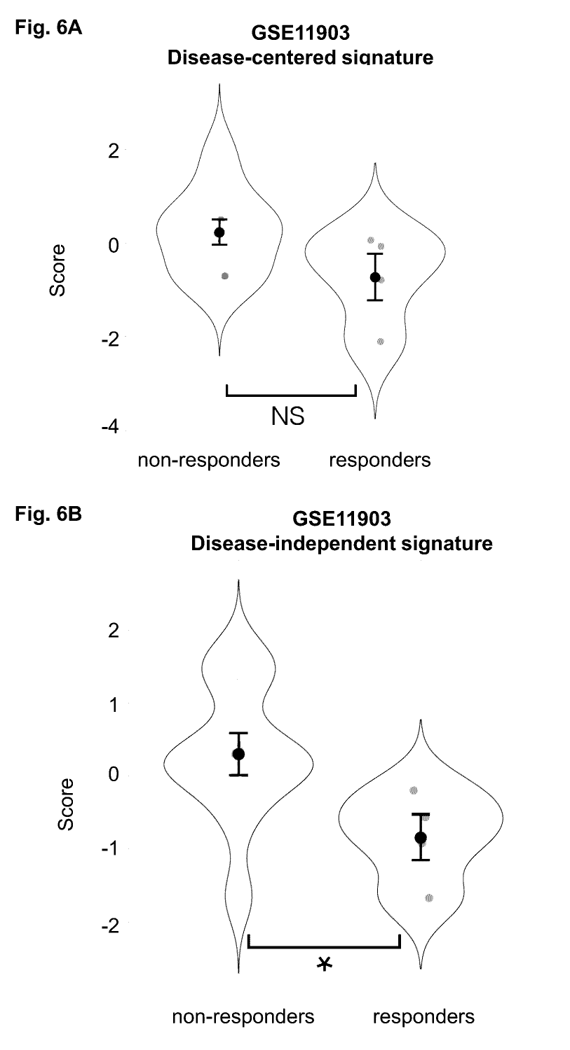

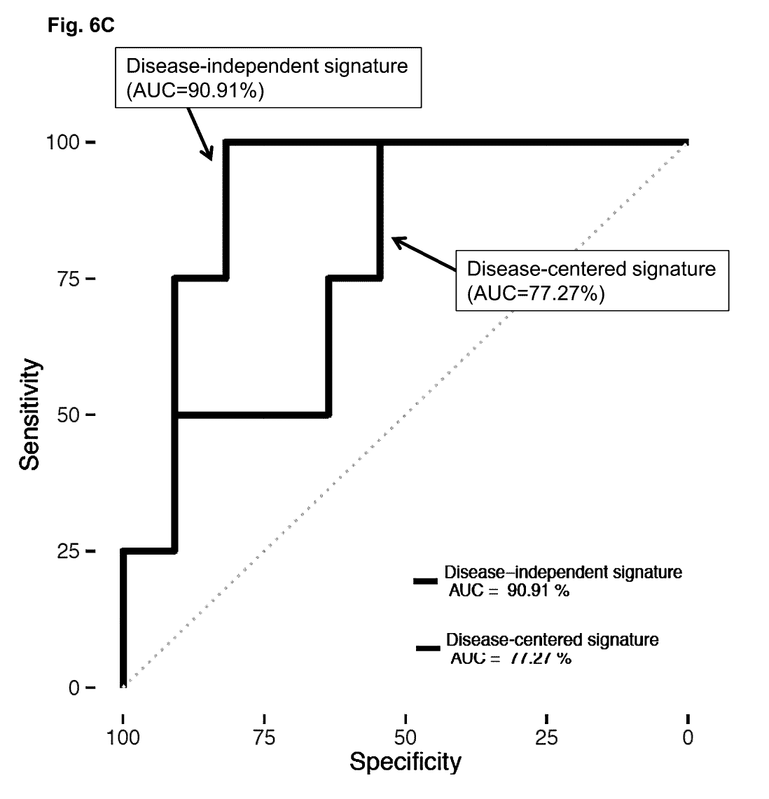

This strategy was then tested on a new validation set profiling expression of skin biopsies in patients affected by Psoriasis before treatment with Etarnecept, a different TNF inhibitor (GSE11903). Despite being structurally different, Etarnecept and Infliximab share the same target and display the same mechanism of action. The disease-centered signature separated responders from non-responders with good but lower performance when compared to IBD patients (AUC=77.27%, FIG. 6A, FIG. 6C). This is to be expected because the original signature was derived from IBD and showed a strong correlation with disease. The disease-independent signature classified responders with much higher accuracy (AUC=90.91%, FIG. 6B, FIG. 6C) and significantly separated responder and non-responder by their signature scores in the validation set (GSE11903, FIG. 6B, p<0.05 t-test).

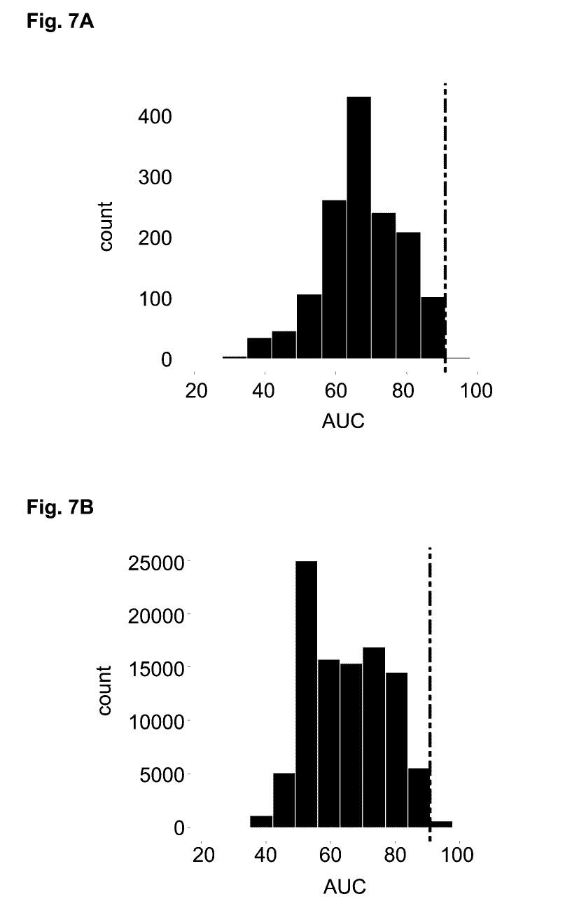

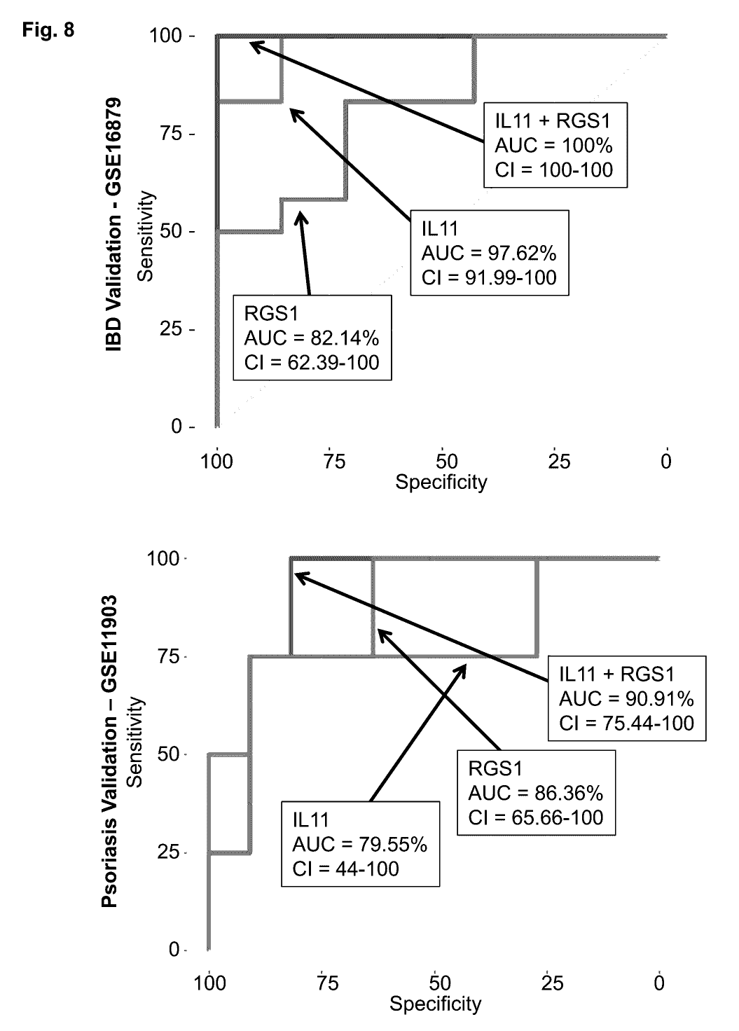

Whether the performance increase of the disease-independent signature could have been observed by chance was next tested. To test this, classification accuracy between responders and non-responders was computed for all possible gene pairs from the disease-centered set and asked whether the disease-independent signature (IL11 and RGS1) was significantly higher than expected by chance. An extensive monte-carlo sampling from all possible gene pairs was also performed. In both cases, the disease-independent signature performed significantly better than random (p-value=0.00140 disease-center set, FIG. 7A; p-value=0.00727 whole genome set, FIG. 7B).

This result indicates that the method used here for separating the disease-independent component of a drug response signature can produce effective biomarkers that are applicable across multiple diseases. The disease-independent signature provided here provides clinicians with an actionable and robust result that can be used to predict whether a patient will benefit from a severe and expensive therapy course. The patient can then be treated appropriately (e.g., with a therapy that includes an anti-TNF agent, or a therapy that does not include an anti-TNF agent).

### Methods

### Meta-Analysis of Gene Expression Data:

Meta-analysis was applied to microarray gene expression data (e.g., as described in Khatri P et al J Exp. Med. 2013). Briefly, an Hedges' g effect size was computed for each gene in each dataset defined as:

$$g = J * \frac{{\tilde{X}}_{1} - {\tilde{X}}_{0}}{\sqrt{\frac{( {n}_{1} - 1 ) {S}_{1}^{2} + ( {n}_{0} - 1 ) {S}_{0}^{2}}{{n}_{1} + {n}_{0} - 2}}}$$

where 1 and 0 represent the group of cases and controls for a given condition respectively. For each gene, a summary effect size was computed using a random effect model as:

$${g}_{s} = \frac{{\sum }_{i}^{n} {W}_{i} {g}_{i}}{{\sum }_{i}^{n} {W}_{i}}$$

where Wᵢ is a weight equal to 1/(Vᵢ+T²), where Vᵢ is the variance of that gene within a given dataset i and T² is the inter-dataset variation (for details: Borenstein M et al Introduction to Meta-analysis, Wiley 2009). For each gene, an FDR was then computed and a final set of genes was selected based on FDR thresholding.

### Computation of a Signature Score:

For a set of signature genes, a signature score was computed as:

$$S = \frac{1}{{n}_{p}} {\sum }_{i}^{{n}_{p}} {log}_{2} {G}_{i} - \frac{1}{{n}_{n}} {\sum }_{j}^{{n}_{n}} {log}_{2} {G}_{j}$$

where nₚ is the subset of positive genes and n_{g} is the subset of negative genes from the signature set of interest (positive indicates an association with cases and negatives with controls). This score was then converted into a z-score as:

$${Z}_{S} = \frac{S - {μ }_{S}}{{σ}_{S}}$$

### Identification of the Disease-Independent Component of a Gene Expression Signature:

A signature for Infliximab response in IBD was computed by using GSE12251 and GSE14580 as discovery cohorts. These datasets measured colon biopsies from patients affected by Ulcerative Colitis prior to drug treatment and healthy control samples. For the purpose of training the model, only samples of IBD patients before Infliximab therapy (baseline) (23 and 30 samples respectively) were selected. Responders were annotated as cases and non-responders as controls and the meta-analysis framework was applied described above.

A signature was then computed for IBD. Because GSE12251 and GSE14580 profiled patients affected by Ulcerative Colitis (UC), a major clinical type of IBD, only datasets profiling patients affected by Ulcerative Colitis compared to Heathy individuals (GSE1152, GSE2461, GSE6731, GSE9686, GSE10191, GSE10616, GSE13367, GSE22619, GSE24287, GSE37283, GSE28713, GSE36807) were uses. UC patients were labeled as cases and healthy individuals as controls.

Genes were selected that were significant in Infliximab response but not in UC (FDR <=5%). This set (FIG. 4a) is defined as I. The original disease-centered signature is defined as D. The intersection between the two sets was performed as:
D∩I
   The result of this intersection was used as the disease-independent signature.

### Example 2

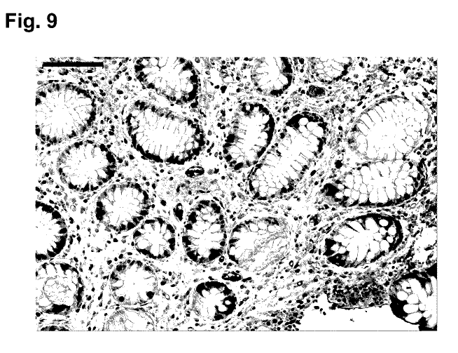

FIG. 9 depicts immunodetection (immunostaining), performed using an anti-RGS1 antibody, of a paraffin embedded colon biopsy from a patient that is responsive to treatment with TNFα. Scale bar is 100 μm. This figure demonstrates that antibody-based methods can be used to detect RGS1 in patients, e.g., those that have inflammatory bowel disease (IBD).

1. A method for predicting whether an individual will respond to treatment with a TNF inhibitor, the method comprising:
(a) measuring an expression level of an RGS1 expression product and an expression level of an IL11 expression product in a biological sample from an individual;
(b) calculating a geometric mean of said measured expression levels to obtain a TNF inhibitor signature score for the individual; and
(c) generating a report that includes the TNF inhibitor signature score and a reference value for the TNF inhibitor signature score.

2. The method according to claim 1, wherein the method comprises, after said generating:
(i) determining that the TNF inhibitor signature score is less than or equal to the reference value, and predicting that the individual will respond to treatment with a TNF inhibitor; or
(ii) determining that the TNF inhibitor signature score is greater than or equal to the reference value, and predicting that the individual will not respond to treatment with a TNF inhibitor.

3. The method according to claim 2, wherein:
the step of determining that the TNF inhibitor signature score is less than or equal to the reference value comprises, after said determining, a step of treating the individual with a TNF inhibitor; and
the step of determining that the TNF inhibitor signature score is greater than or equal to the reference value comprises, after said determining, a step of treating the individual with a therapy that does not include administration of a TNF inhibitor.

4. The method according to claim 1, wherein:
(i) the method comprises, after said calculating, determining that the TNF inhibitor signature score is less than or equal to the reference value, wherein said report comprises a prediction that the individual will respond to treatment with a TNF inhibitor; or
(ii) the method comprises, after said calculating, determining that the TNF inhibitor signature score is greater than or equal to the reference value, wherein said report comprises a prediction that the individual will not respond to treatment with a TNF inhibitor.

5. The method according to claim 1, wherein:
the method comprises, after said calculating: (i) determining that the TNF inhibitor signature score is less than or equal to the reference value, and (ii) treating the individual with a TNF inhibitor; or
the method comprises, after said calculating: (i) determining that the TNF inhibitor signature score is greater than or equal to the reference value; and (ii) treating the individual with a therapy that does not include administration of a TNF inhibitor.

6. The method according to claim 1, wherein the biological sample is a biopsy.

7. The method according to claim 1, wherein the individual has an inflammatory bowel disease and/or psoriasis.

8. (canceled)

9. The method according to claim 1, wherein the RGS1 expression product is an mRNA encoding RGS1 and the IL11 expression product is an mRNA encoding IL11.

10. The method according to claim 9, wherein said measuring comprises an assay selected from: quantitative RT-PCR, microarray, and nucleic acid sequencing.

11. The method according to claim 1, wherein the RGS1 and IL11 expression products are proteins.

12. The method according to claim 11, wherein said measuring comprises an assay selected from: ELISA, Western blot, mass spectrometry, and flow cytometry.

13. A method of treating an individual in need thereof, the method comprising:
(a) measuring an expression level of an RGS1 expression product and an expression level of an IL11 expression product in a biological sample from an individual;
(b) calculating the geometric mean of said measured expression levels to obtain a TNF inhibitor signature score; and either
(i) determining that the TNF inhibitor signature score is less than or equal to a reference value, and treating the individual with a TNF inhibitor; or
(ii) determining that the TNF inhibitor signature score is greater than or equal to a reference value, and treating the individual with a therapy that does not include administration of a TNF inhibitor.

14. The method according to claim 13, wherein the biological sample is a biopsy.

15. The method according to claim 13, wherein the individual has an inflammatory bowel disease and/or psoriasis.

16. (canceled)

17. The method according to claim 13, wherein the RGS1 expression product is an mRNA encoding RGS1 and the IL11 expression product is an mRNA encoding IL11; and wherein said measuring comprises an assay selected from: quantitative RT-PCR, microarray, and nucleic acid sequencing.

18. The method according to claim 13, wherein the RGS1 and IL11 expression products are proteins; and wherein said measuring comprises an assay selected from: ELISA, Western blot, mass spectrometry, and flow cytometry.

19. A method of treating an individual with inflammatory bowel disease and/or psoriasis, the method comprising:
measuring an expression level of an RGS1 expression product in a biological sample from the individual, and either
(i) determining that said expression is less than or equal to a reference value, and treating the individual with a TNF inhibitor, or
(ii) determining that said expression is greater than or equal to a reference value, and treating the individual with a therapy that does not include administration of a TNF inhibitor.

20. The method according to claim 19, wherein the biological sample is a biopsy.

21. The method according to claim 19, wherein the RGS1 expression product is an mRNA encoding RGS1 and said measuring comprises an assay selected from: quantitative RT-PCR, microarray, and nucleic acid sequencing.

22. The method according to claim 19, wherein the RGS1 expression product is a protein and said measuring comprises an assay selected from: ELISA, Western blot, mass spectrometry, and flow cytometry.

23-24. (canceled)


SEQUENCE LISTING (6 sequences)

SEQ ID NO: 1 - protein, 209 aa, Homo sapiens
MRAAAISTPKLDKMPGMFFSANPKELKGTTHSLLDDKMQKRRPKTFGMDMKAYLRSMIPHLESGMKSSKSKDVLSAAEVMQWSQSLEKLLANQTGQNVFGSFLKSEFSEENIEFWLACEDYKKTESDLLPCKAEEIYKAFVHSDAAKQINIDFRTRESTAKKIKAPTPTCFDEAQKVIYTLMEKDSYPRFLKSDIYLNLLNDLQANSLK

SEQ ID NO: 2 - DNA, 1403 bp, Homo sapiens
gcctgtctgcattctactatataaagcagcagagacgttgactagcgcatatttgctaagagcaccatgcgcgcagcagccatctccactccaaagttagacaaaatgccaggaatgttcttctctgctaacccaaaggaattgaaaggaaccactcattcacttctagacgacaaaatgcaaaaaaggaggccaaagacttttggaatggatatgaaagcatacctgagatctatgatcccacatctggaatctggaatgaaatcttccaagtccaaggatgtactttctgctgctgaagtaatgcaatggtctcaatctctggaaaaacttcttgccaaccaaactggtcaaaatgtctttggaagtttcctaaagtctgaattcagtgaggagaatattgagttctggctggcttgtgaagactataagaaaacagagtctgatcttttgccctgtaaagcagaagagatatataaagcatttgtgcattcagatgctgctaaacaaatcaatattgacttccgcactcgagaatctacagccaagaagattaaagcaccaacccccacgtgttttgatgaagcacaaaaagtcatatatactcttatggaaaaggactcttatcccaggttcctcaaatcagatatttacttaaatcttctaaatgacctgcaggctaatagcctaaagtgactggtccctggctgaagggaattaacagatagtatcaagcgcagaaggaatgtgccagtatggctccctgggtgaacagcttggccttttttgggtgtcttgacaggccaagaagaacaaatgactcagaatggattaacatgaaagttatccaggcgcagagttgaagaagcataagcaagacaaaaacagagagaccgcagaaggaggaagatactgtggtactgtcataaaaaacagtggagctctgtattagaaagcccctcagaactgggaaggccaggtaactctagttacacagaaactgtgactaaagtctatgaaactgattacaacagactgtaagaatcaaagtcaactgacatctatgctacatattattatatagtttgtactgagctattgaagtcccattaacttaaagtatatgttttcaaattgccattgctactattgcttgtcggtgttattttattttattgtttttgactttggaagagatgaactgtgtatttaacttaagctattgctcttaaaaccagggagtcagaatatatttgtaagttaaatcattggtgctaataataaatgtggattttgtattaaaatatatagaagcaatttctgtttacatgtccttgctacttttaaaaacttgcatttattcctcagattttaaaaataaataaataattcatttaagattc

SEQ ID NO: 3 - protein, 199 aa, Homo sapiens
MNCVCRLVLVVLSLWPDTAVAPGPPPGPPRVSPDPRAELDSTVLLTRSLLADTRQLAAQLRDKFPADGDHNLDSLPTLAMSAGALGALQLPGVLTRLRADLLSYLRHVQWLRRAGGSSLKTLEPELGTLQARLDRLLRRLQLLMSRLALPQPPPDPPAPPLAPPSSAWGGIRAAHAILGGLHLTLDWAVRGLLLLKTRL

SEQ ID NO: 4 - protein, 120 aa, Homo sapiens
MSAGALGALQLPGVLTRLRADLLSYLRHVQWLRRAGGSSLKTLEPELGTLQARLDRLLRRLQLLMSRLALPQPPPDPPAPPLAPPSSAWGGIRAAHAILGGLHLTLDWAVRGLLLLKTRL

SEQ ID NO: 5 - DNA, 2381 bp, Homo sapiens
actgccgcggccctgctgctcagggcacatgcctcccctccccaggccgcggcccagctgaccctcggggctcccccggcagcggacagggaagggttaaaggcccccggctccctgccccctgccctggggaacccctggccctgtggggacatgaactgtgtttgccgcctggtcctggtcgtgctgagcctgtggccagatacagctgtcgcccctgggccaccacctggcccccctcgagtttccccagaccctcgggccgagctggacagcaccgtgctcctgacccgctctctcctggcggacacgcggcagctggctgcacagctgagggacaaattcccagctgacggggaccacaacctggattccctgcccaccctggccatgagtgcgggggcactgggagctctacagctcccaggtgtgctgacaaggctgcgagcggacctactgtcctacctgcggcacgtgcagtggctgcgccgggcaggtggctcttccctgaagaccctggagcccgagctgggcaccctgcaggcccgactggaccggctgctgcgccggctgcagctcctgatgtcccgcctggccctgccccagccacccccggacccgccggcgcccccgctggcgcccccctcctcagcctgggggggcatcagggccgcccacgccatcctgggggggctgcacctgacacttgactgggccgtgaggggactgctgctgctgaagactcggctgtgacccggggcccaaagccaccaccgtccttccaaagccagatcttatttatttatttatttcagtactgggggcgaaacagccaggtgatccccccgccattatctccccctagttagagacagtccttccgtgaggcctggggggcatctgtgccttatttatacttatttatttcaggagcaggggtgggaggcaggtggactcctgggtccccgaggaggaggggactggggtcccggattcttgggtctccaagaagtctgtccacagacttctgccctggctcttccccatctaggcctgggcaggaacatatattatttatttaagcaattacttttcatgttggggtggggacggaggggaaagggaagcctgggtttttgtacaaaaatgtgagaaacctttgtgagacagagaacagggaattaaatgtgtcatacatatccacttgagggcgatttgtctgagagctggggctggatgcttgggtaactggggcagggcaggtggaggggagacctccattcaggtggaggtcccgagtgggcggggcagcgactgggagatgggtcggtcacccagacagctctgtggaggcagggtctgagccttgcctggggccccgcactgcatagggccttttgtttgttttttgagatggagtctcgctctgttgcctaggctggagtgcagtgaggcaatctgaggtcactgcaacctccacctcccgggttcaagcaattctcctgcctcagcctcccgattagctgggatcacaggtgtgcaccaccatgcccagctaattatttatttcttttgtatttttagtagagacagggtttcaccatgttggccaggctggtttcgaactcctgacctcaggtgatcctcctgcctcggcctcccaaagtgctgggattacaggtgtgagccaccacacctgacccataggtcttcaataaatatttaatggaaggttccacaagtcaccctgtgatcaacagtacccgtatgggacaaagctgcaaggtcaagatggttcattatggctgtgttcaccatagcaaactggaaacaatctagatatccaacagtgagggttaagcaacatggtgcatctgtggatagaacgccacccagccgcccggagcagggactgtcattcagggaggctaaggagagaggcttgcttgggatatagaaagatatcctgacattggccaggcatggtggctcacgcctgtaatcctggcactttgggaggacgaagcgagtggatcactgaagtccaagagttcgagaccggcctgcgagacatggcaaaaccctgtctcaaaaaagaaagaatgatgtcctgacatgaaacagcaggctacaaaaccactgcatgctgtgatcccaattttgtgtttttctttctatatatggattaaaacaaaaatcctaaagggaaatacgccaaaatgttgacaatgactgtctccaggtcaaaggagagaggtgggattgtgggtgacttttaatgtgtatgattgtctgtattttacagaatttctgccatgactgtgtattttgcatgacacattttaaaaataataaacactatttttagaataacagaaaaa

SEQ ID NO: 6 - DNA, 2208 bp, Homo sapiens
actgccgcggccctgctgctcagggcacatgcctcccctccccaggccgcggcccagctgaccctcggggctcccccggcagcggacagggaagggttaaaggcccccggctccctgccccctgccctggggaacccctggccctgtggggacatgaactagggacaaattcccagctgacggggaccacaacctggattccctgcccaccctggccatgagtgcgggggcactgggagctctacagctcccaggtgtgctgacaaggctgcgagcggacctactgtcctacctgcggcacgtgcagtggctgcgccgggcaggtggctcttccctgaagaccctggagcccgagctgggcaccctgcaggcccgactggaccggctgctgcgccggctgcagctcctgatgtcccgcctggccctgccccagccacccccggacccgccggcgcccccgctggcgcccccctcctcagcctgggggggcatcagggccgcccacgccatcctgggggggctgcacctgacacttgactgggccgtgaggggactgctgctgctgaagactcggctgtgacccggggcccaaagccaccaccgtccttccaaagccagatcttatttatttatttatttcagtactgggggcgaaacagccaggtgatccccccgccattatctccccctagttagagacagtccttccgtgaggcctggggggcatctgtgccttatttatacttatttatttcaggagcaggggtgggaggcaggtggactcctgggtccccgaggaggaggggactggggtcccggattcttgggtctccaagaagtctgtccacagacttctgccctggctcttccccatctaggcctgggcaggaacatatattatttatttaagcaattacttttcatgttggggtggggacggaggggaaagggaagcctgggtttttgtacaaaaatgtgagaaacctttgtgagacagagaacagggaattaaatgtgtcatacatatccacttgagggcgatttgtctgagagctggggctggatgcttgggtaactggggcagggcaggtggaggggagacctccattcaggtggaggtcccgagtgggcggggcagcgactgggagatgggtcggtcacccagacagctctgtggaggcagggtctgagccttgcctggggccccgcactgcatagggccttttgtttgttttttgagatggagtctcgctctgttgcctaggctggagtgcagtgaggcaatctgaggtcactgcaacctccacctcccgggttcaagcaattctcctgcctcagcctcccgattagctgggatcacaggtgtgcaccaccatgcccagctaattatttatttcttttgtatttttagtagagacagggtttcaccatgttggccaggctggtttcgaactcctgacctcaggtgatcctcctgcctcggcctcccaaagtgctgggattacaggtgtgagccaccacacctgacccataggtcttcaataaatatttaatggaaggttccacaagtcaccctgtgatcaacagtacccgtatgggacaaagctgcaaggtcaagatggttcattatggctgtgttcaccatagcaaactggaaacaatctagatatccaacagtgagggttaagcaacatggtgcatctgtggatagaacgccacccagccgcccggagcagggactgtcattcagggaggctaaggagagaggcttgcttgggatatagaaagatatcctgacattggccaggcatggtggctcacgcctgtaatcctggcactttgggaggacgaagcgagtggatcactgaagtccaagagttcgagaccggcctgcgagacatggcaaaaccctgtctcaaaaaagaaagaatgatgtcctgacatgaaacagcaggctacaaaaccactgcatgctgtgatcccaattttgtgtttttctttctatatatggattaaaacaaaaatcctaaagggaaatacgccaaaatgttgacaatgactgtctccaggtcaaaggagagaggtgggattgtgggtgacttttaatgtgtatgattgtctgtattttacagaatttctgccatgactgtgtattttgcatgacacattttaaaaataataaacactatttttagaataacagaaaaa